(19) United States
(12) Patent Application Publication
(10) Pub. No.: US 2023/0381542 A1
(43) Pub. Date: Nov. 30, 2023

(54) SYSTEMS AND METHODS FOR QUALITY ASSURANCE

(71) Applicant: SHANGHAI UNITED IMAGING HEALTHCARE CO., LTD., Shanghai (CN)

(72) Inventors: Cheng NI, Shanghai (CN); Yanfang LIU, Shanghai (CN); Wei ZHANG, Shanghai (CN); Li WANG, Shanghai (CN); Yifeng WANG, Shanghai (CN); Jingjie ZHOU, Shanghai (CN); Fei GONG, Shanghai (CN); Fei ZHAO, Shanghai (CN); Shaoqiang YE, Shanghai (CN); Binghuan LI, Shanghai (CN); Jiaqi FU, Shanghai (CN); Can LIAO, Shanghai (CN); Feichao FU, Shanghai (CN)

(73) Assignee: SHANGHAI UNITED IMAGING HEALTHCARE CO., LTD., Shanghai (CN)

(21) Appl. No.: 18/327,050

(22) Filed: May 31, 2023

(30) Foreign Application Priority Data
May 31, 2022 (CN) 202210611450.9
Sep. 29, 2022 (WO) PCT/CN2022/122413
Sep. 29, 2022 (WO) PCT/CN2022/122415

(51) Int. Cl. A61N 5/10; G16H 40/40

(52) U.S. Cl. CPC A61N 5/1075; A61N 5/1071; G16H 40/40

(57) ABSTRACT

A method for quality assurance may include obtaining a state of a medical device. The method may also include obtaining a target plan of a target subject. The method may also include determining a prediction result based on the state of the medical device and the target plan of the target subject. The method may also include determining whether a quality assurance test passes based on the prediction result.

### CROSS-REFERENCE TO RELATED APPLICATIONS

This application claims priority of International Application No. PCT/CN2022/122415, filed on Sep. 29, 2022, International Application No. PCT/CN2022/122413, filed on Sep. 29, 2022, and Chinese Patent Application No. 202210611450.9, filed on May 31, 2022, the contents of each of which are hereby incorporated by reference.

### TECHNICAL FIELD

The present disclosure generally relates to systems and methods for medical imaging, and more particularly, relates to systems and methods for quality assurance in the medical imaging.

### BACKGROUND

Radiotherapy (RT) is widely used in clinical treatment for cancers and other conditions of a patient. In order to ensure an accurate implementation of a radiotherapy plan of a patient, it is necessary to perform a quality assurance test on the radiotherapy plan and/or an RT device. However, the quality assurance test may be time-consuming and inefficient. In addition, when the quality assurance test does not pass, it is difficult to determine a reason that the quality assurance test does not pass. Therefore, it is desirable to provide systems and methods for quality assurance, thereby improving the accuracy and/or efficiency of subsequent medical operations.

### SUMMARY

According to a first aspect of the present disclosure, a method for quality assurance may include one or more of the following operations. One or more processors may obtain a state of a medical device. The one or more processors may obtain a target plan of a target subject. The one or more processors may determine a prediction result based on the state of the medical device and the target plan of the target subject. The one or more processors may determine whether a quality assurance test passes based on the prediction result.

In some embodiments, the state of the medical device may include at least one of beam information corresponding to a plurality of dose rates, positioning accuracy information of at least one component of the medical device, or operation error information of the at least one component of the medical device.

In some embodiments, to obtain a state of a medical device, the one or more processors may obtain a data set. The data set may be determined based on at least one of at least one candidate plan, limit performance information of at least one component of the medical device, or a calculation limit of a dose model. The one or more processors may obtain the state of the medical device by directing the medical device to execute the data set.

In some embodiments, the data set may include at least one of a first data set, a second data set, or a third data set. To obtain a data set, the one or more processors may determine the first data set based on at least one first candidate plan, wherein a value of a first candidate parameter in the at least one first candidate plan is within a first range. The one or more processors may determine the second data set based on at least one second candidate plan, wherein a value of a second candidate parameter in the at least one second candidate plan is outside a second range. Or the one or more processors may determine the third data set based on at least one of the limit performance information of the at least one component of the medical device or the calculation limit of the dose model.

In some embodiments, the data set may include at least one sample parameter, each of which corresponds to at least one sample parameter value. To obtain the state of the medical device by directing the medical device to execute the data set, the one or more processors may determine an actual test result related to at least one of the target plan of the target subject or the medical device by directing the medical device to execute the data set. The target plan may include at least one target parameter, each of which corresponds to at least one target parameter value. The one or more processors may determine a data set execution result based on the actual test result. The one or more processors may obtain the state of the medical device based on the data set execution result.

In some embodiments, to determine a prediction result based on the state of the medical device and the target plan of the target subject, the one or more processors may determine at least one of feature information related to a complexity level of the target plan or a target fluence map based on the target plan of the target subject. The one or more processors may determine the prediction result based on the state of the medical device and the at least one of the feature information related to the complexity level of the target plan or the target fluence map.

In some embodiments, the target subject may include a plurality of regions of interest (ROIs). The prediction result may include dose distributions corresponding to the plurality of ROIs respectively. To determine whether a quality assurance test passes based on the prediction result, the one or more processors may determine a weight corresponding to each ROI of the plurality of ROIs. The one or more processors may determine whether the quality assurance test passes based on the weights and the dose distributions corresponding to the plurality of ROIs respectively.

In some embodiments, to determine a prediction result based on the state of the medical device and the target plan of the target subject, the one or more processors may determine the prediction result based on the state of the medical device and the target plan of the target subject using a first model. The first model may be a machine learning model. The prediction result may include at least one of a predicted image of the target subject, a gamma passing rate, or a dose distribution.

In some embodiments, in response to determining that the quality assurance test does not pass based on the prediction result, the one or more processors may determine a reason that the quality assurance test does not pass based on the state of the medical device, the target plan of the target subject, and the prediction result using a second model.

In some embodiments, the one or more processors may adjust, based on the reason that the quality assurance test does not pass, a value of a parameter associated with at least one of the medical device, the target plan, or a dose model.

In some embodiments, the one or more processors may determine an updated prediction result based on an adjusted value of the parameter using the first model. The one or more processors may determine whether the quality assurance test passes based on the updated prediction result. In response to determining that the quality assurance test passes, the one or more processors may control the medical device to treat and/or scan the target subject according to an updated target plan. The updated target plan may be determined based on the adjusted value of the parameter.

According to another aspect of the present disclosure, a method for quality assurance may include one or more of the following operations. One or more processors may obtain a state of a medical device. The one or more processors may obtain a target plan of a target subject. The one or more processors may determine a prediction result based on the state of the medical device and the target plan of the target subject using a quality assurance model. The quality assurance model may be a machine learning model.

According to yet another aspect of the present disclosure, a method for quality assurance may include one or more of the following operations. One or more processors may obtain a data set for quality assurance. The data set may include at least one sample parameter, each of which corresponds to at least one sample parameter value. The one or more processors may determine an actual test result related to at least one of a target plan of a target subject or a medical device based on the data set. The target plan may include at least one target parameter, each of which corresponds to at least one target parameter value. The one or more processors may determine a quality assurance result related to the at least one of the target plan or the medical device based on the actual test result.

In some embodiments, to determine a quality assurance result related to the at least one of the target plan or the medical device based on the actual test result, the one or more processors may determine a predicted test result based on the data set using a test model. The one or more processors may determine the quality assurance result based on the predicted test result and the actual test result.

In some embodiments, the actual test result may include a test image obtained by the medical device based on the data set. The predicted test result may include a simulated image obtained based on the test model. To determine the quality assurance result based on the predicted test result and the actual test result, the one or more processors may determine the quality assurance result based on a difference between the test image and the simulated image.

In some embodiments, the data set may be determined based on at least one of a plurality of sample plans or limit performance information of at least one component of the medical device.

In some embodiments, the plurality of sample plans may comprise plans corresponding to different complexity levels.

In some embodiments, the one or more processors may determine whether a complexity level of the target plan is within a complexity range of the data set. The complexity range of the data set may be determined based on the complexity level of the at least one of the plurality of sample plans corresponding to the data set. In response to determining that the complexity level of the target plan is within the complexity range, the one or more processors may control the medical device to perform a medical operation on the target subject based on the target plan. In response to determining that the complexity level of the target plan is not within the complexity range, the one or more processors may proceed with at least one of the following operations. The one or more processors may adjust the data set based on the target plan. The one or more processors may perform a quality assurance test on the target plan.

In some embodiments, the data set may include a plurality of data subsets. The plurality of data subsets may be configured to correspond to at least one of: different quality assurance test frequencies or different types of plans.

In some embodiments, the one or more processors may determine whether a quality assurance test passes based on the quality assurance result. In response to determining that the quality assurance test does not pass, the one or more processors may adjust at least one of a parameter of the medical device or the sample parameter value of the data set.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a schematic diagram illustrating exemplary hardware and/or software components of an exemplary computing device on which a processing device may be implemented according to some embodiments of the present disclosure;

FIG. 3 is a schematic diagram illustrating exemplary hardware and/or software components of an exemplary mobile device according to some embodiments of the present disclosure;

FIG. 4 is a schematic diagram illustrating an exemplary processing device according to some embodiments of the present disclosure;

FIG. 5 is a flowchart illustrating an exemplary process for determining whether a quality assurance test passes according to some embodiments of the present disclosure;

FIG. 6A is a flowchart illustrating an exemplary process for obtaining a state of a medical device according to some embodiments of the present disclosure;

FIG. 6B is a schematic diagram illustrating an exemplary first data set, an exemplary second data set, and an exemplary third data set according to some embodiments of the present disclosure;

FIG. 7 is a flowchart illustrating an exemplary process for obtaining a state of a medical device according to some embodiments of the present disclosure;

FIG. 8 is a flowchart illustrating an exemplary process for determining a prediction result according to some embodiments of the present disclosure;

FIG. 9 is a flowchart illustrating an exemplary process for performing a quality assurance test according to some embodiments of the present disclosure;

FIG. 10 is a flowchart illustrating an exemplary process for determining a first model and a second model according to some embodiments of the present disclosure;

FIG. 11 is a schematic diagram illustrating an exemplary processing device according to some embodiments of the present disclosure;

FIG. 12 is a flowchart illustrating an exemplary process for determining a prediction result according to some embodiments of the present disclosure;

FIG. 13 is a schematic diagram illustrating an exemplary processing device according to some embodiments of the present disclosure;

FIG. 14 is a flowchart illustrating an exemplary process for determining a quality assurance result according to some embodiments of the present disclosure; and

FIG. 15 is a flowchart illustrating an exemplary process for performing a quality assurance test according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections, or assembly of different level in ascending order. However, the terms may be displaced by another expression if they achieve the same purpose.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “anatomical structure” in the present disclosure may refer to gas (e.g., air), liquid (e.g., water), solid (e.g., stone), cell, tissue, organ of a subject, or any combination thereof, which may be displayed in an image and really exist in or on the subject's body. The term “region,” “location,” and “area” in the present disclosure may refer to a location of an anatomical structure shown in the image or an actual location of the anatomical structure existing in or on the subject's body, since the image may indicate the actual location of a certain anatomical structure existing in or on the subject's body.

Radiotherapy refers to the use of radiation to treat tumors. During a radiotherapy operation of a patient, due to the high beam energy, normal tissues of the patient may also be affected while tumor tissues of the patient are killed. In order to minimize the damage to the normal tissues, it is necessary to determine a radiotherapy plan. In order to ensure the accurate implementation of the radiotherapy plan, it is necessary to perform a quality assurance test for a medical device and/or the radiotherapy plan. The quality assurance test for the medical device may refer to a test process of an operation state (e.g., a function, a control accuracy) of at least one component (e.g., an accelerator) of the medical device. The quality assurance test for the radiotherapy plan may refer to a test process of radiotherapy parameter(s) associated with a radiotherapy process of a patient performed according to the radiotherapy plan.

Generally, in an existing quality assurance test, a measured dose distribution may be measured using a device (e.g., a film dosimeter, an ionization chamber, an electronic field imaging device (EPID)) according to radiotherapy parameters in the radiotherapy plan of the patient, and a calculated dose distribution may be determined by a radiation treatment plan system (TPS). Then a gamma passing rate may be determined by comparing the measured dose distribution with the calculated dose distribution using a gamma analysis method. However, the existing method may be expensive and time-consuming, and the quality assurance test has to be performed for each patient's radiotherapy plan. In addition, it is difficult to determine a reason that the quality assurance test does not pass. Furthermore, in an online radiotherapy process (e.g., an online adaptive radiotherapy), there are usually no conditions for performing the quality assurance test.

Accordingly, an aspect of the present disclosure relates to systems and methods for quality assurance. According to some embodiments of the present disclosure, a processing device may obtain a state of a medical device (e.g., beam information corresponding to a plurality of dose rates, positioning accuracy information of at least one component of the medical device, operation error information of the at least one component of the medical device). The processing device may also obtain a target plan of a target subject. Further, the processing device may determine a prediction result (e.g., a predicted image of the target subject, a gamma passing rate, a dose distribution) based on the state of the medical device and the target plan of the target subject. For example, the processing device may determine the prediction result based on the state of the medical device and the target plan of the target subject using a first model (e.g., a machine learning model). The processing device may determine whether a quality assurance test passes based on the prediction result. In response to determining that the quality assurance test passes, the processing device may control the medical device to treat and/or scan the target subject according to the target plan. In response to determining that the quality assurance test does not pass, the processing device may determine a reason that the quality assurance test does not pass based on the state of the medical device, the target plan of the target subject, and the prediction result using a second model (e.g., a machine learning model).

The methods and systems disclosed herein can improve the rationality, accuracy, and efficiency of the quality assurance test by, for example, reducing the workload of a user, cross-user variations, and/or the time needed for the quality assurance test. In addition, when the prediction result satisfies a preset condition, the processing device may determine that the quality assurance test passes, and the medical device may be controlled to treat and/or scan the target subject according to the target plan, without performing a quality assurance test for the target plan using the medical device, thereby simplifying the radiotherapy process. In addition, the reason that the quality assurance test does not pass and a target adjustment mode may be determined based on the prediction result, which can improve the efficiency of the radiotherapy process.

Further, another aspect of the present disclosure relates to systems and methods for quality assurance. According to some embodiments of the present disclosure, a processing device may obtain a data set. The data set may include at least one sample parameter, each of which corresponds to at least one sample parameter value. The processing device may determine an actual test result related to at least one of a target plan of a target subject or a medical device based on the data set. The target plan may include at least one target parameter, each of which corresponds to at least one target parameter value. Then the processing device may determine a quality assurance result related to at least one of the target plan or the medical device based on the actual test result and determine whether a quality assurance test passes based on the quality assurance result.

According to the methods and systems of the present disclosure, the quality assurance test may be performed by directing the medical device to execute the data set according to a certain test frequency (e.g., once a day, once a week, once a month, once a year). Since the data set is determined based on a plurality of sample plans with relatively high complexity levels, ideally, the complexity level of the data set may be higher than complexity levels of plans of almost all patients. In other words, if the quality assurance result determined based on the data set indicates that the quality assurance test passes, it may indicate that a quality assurance test based on a plan with a relatively low complexity level can also pass. Therefore, it is no longer necessary to perform the quality assurance test for each patient's plan before the radiotherapy operation of the patient, which can simplify the quality assurance test and improve the efficiency of the quality assurance test. In other words, before a radiotherapy operation of a patient, the quality assurance result of the data set is checked to replace the operation of performing a quality assurance test for the patient's plan.

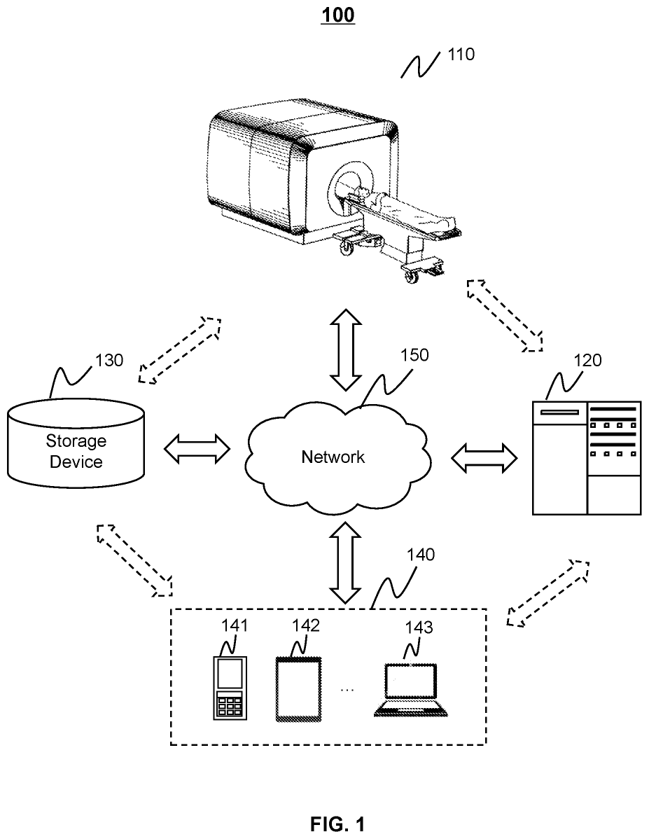

FIG. 1 is a schematic diagram illustrating an exemplary medical system according to some embodiments of the present disclosure. As illustrated, a medical system 100 may include a medical device 110, a processing device 120, a storage device 130, a terminal device 140, and a network 150. The components of the medical system 100 may be connected in one or more of various ways. Merely by way of example, as illustrated in FIG. 1, the medical device 110 may be connected to the processing device 120 directly as indicated by the bi-directional arrow in dotted lines linking the medical device 110 and the processing device 120, or through the network 150. As another example, the storage device 130 may be connected to the medical device 110 directly as indicated by the bi-directional arrow in dotted lines linking the medical device 110 and the storage device 130, or through the network 150. As still another example, the terminal device 140 may be connected to the processing device 120 directly as indicated by the bi-directional arrow in dotted lines linking the terminal device 140 and the processing device 120, or through the network 150. As still another example, the terminal device 140 may be connected to the storage device 130 directly as indicated by the bi-directional arrow in dotted lines linking the terminal device 140 and the storage device 130, or through the network 150.

The medical device 110 may be configured to acquire imaging data relating to a subject (also referred to as a target subject in the present disclosure) and/or perform a radiotherapy treatment on the subject. The imaging data relating to a subject may include an image (e.g., an image slice), projection data, or a combination thereof. In some embodiments, the imaging data may be two-dimensional (2D) imaging data, three-dimensional (3D) imaging data, four-dimensional (4D) imaging data, or the like, or any combination thereof. The subject may include any biological subject (e.g., a human being, an animal, a plant, or a portion thereof) and/or a non-biological subject (e.g., a phantom, structure/device to be non-destructively tested).

In some embodiments, the medical device 110 may be an RT device. The RT device may be configured to deliver a radiotherapy treatment. For example, the RT device may deliver one or more radiation beams to a target (e.g., a tumor) of a subject (e.g., a patient) for causing an alleviation of the subject's disease and/or symptoms.

In some embodiments, the medical device 110 may include a single modality imaging device. For example, the medical device 110 may include an X-ray therapy device, a Co-60 teletherapy device, a medical electron accelerator, or the like. In some embodiments, the medical device 110 may include a multi-modality imaging device configured to obtain medical images related to the subject and perform a radiotherapy operation on the subject. In some embodiments, the medical device 110 may include an image guided radiation therapy (IGRT) device. For example, the medical device 110 may include a computed tomography (CT) guided radiotherapy device, a magnetic resonance imaging (MRI) guided radiotherapy device, or the like. It should be noted that the medical device described above is merely provided for illustration purposes, and not intended to limit the scope of the present disclosure. The term “imaging modality” or “modality” as used herein broadly refers to an imaging method or technology that gathers, generates, processes, and/or analyzes imaging information of a subject.

In some embodiments, the medical device 110 may include a treatment component (e.g., a treatment head). The treatment component may be configured to perform a radiation operation on the subject. The treatment component may include a treatment head and a gantry. The treatment head may be connected with the gantry, and the treatment head may move with a motion (e.g., rotation) of the gantry. In some embodiments, the treatment head may include a target, a treatment radiation source, a collimator, etc. The treatment radiation source may be configured to generate and emit a radiation beam toward the subject for treatment. In some embodiments, the radiation beam emitted by the treatment radiation source may include electrons, photons, or other types of radiation. In some embodiments, the treatment radiation source may be an MV radiation source for emitting an MV beam or a kilovoltage (kV) radiation source for emitting a kV beam (i.e., a radiation beam whose energy is within the kilovoltage range (e.g., >1 keV)). In some embodiments, the treatment radiation source may include a linear accelerator (LINAC) configured to accelerate electrons, ions, or protons. The collimator may be configured to control a shape of the radiation beam generated by the treatment radiation source. In some embodiments, the collimator may include an aperture, a primary collimator, a secondary collimator, etc. The secondary collimator may include a multi-leaf collimator and a tungsten gate. The multi-leaf collimator may include a plurality of leaves. In some embodiments, the positions of the plurality of leaves and/or the tungsten gate may form a radiation area (also referred to as a radiation field). In some embodiments, the plurality of leaves may be driven by one or more driving components (e.g., motors) to move to specific positions to change a shape of the radiation field.

In some embodiments, the medical device 110 may include a radiotherapy auxiliary device, such as an electronic field imaging device (EPID). The EPID may generate an image of the subject prior to the radiation operation, during the radiation operation, and/or after the radiation operation. The EPID may include a detector for detecting the radiation emitted from the treatment radiation source. In some embodiments, the detector may include one or more detector units. The detector unit(s) may include a scintillation detector (e.g., a cesium iodide detector, a gadolinium oxysulfide detector), a gas detector, etc. The detector unit(s) may include a single-row detector and/or a multi-row detector.

In some embodiments, the medical device 110 may include an imaging device. The imaging device may be configured to perform an imaging operation prior to the radiation operation, during the radiation operation, and/or after the radiation operation. In some embodiments, the imaging device may include an X-ray imaging device, a CT device (e.g., a 3D CT device, a 4D CT device), or the like, or any combination thereof. In some embodiments, the imaging device may include a cone beam computed tomography (CBCT) device, a multislice computed tomography (MSCT) device, a fan-beam computed tomography (FBCT) device, or the like, or any combination thereof. In some embodiments, the imaging device may include an ultrasound imaging device, a fluoroscopy imaging device, a magnetic resonance imaging (MRI) device, a single photon emission computed tomography (SPECT) device, a positron emission tomography (PET) device, or the like, or any combination thereof. In some embodiments, the imaging device may include an imaging radiation source, a detector, or the like. In some embodiments, the imaging radiation source and the treatment radiation source may be integrated into a single radiation source for imaging and/or treatment of the subject.

The processing device 120 may process data and/or information obtained from the medical device 110, the storage device 130, and/or the terminal device 140. For example, the processing device 120 may obtain a state of a medical device and a target plan of a target subject; the processing device 120 may determine a prediction result based on a state of the medical device and a target plan of a target subject; then the processing device 120 may determine whether a quality assurance test passes based on the prediction result. As another example, the processing device 120 may obtain a data set and determine an actual test result related to at least one of a target plan of a target subject or a medical device based on the data set; then the processing device 120 may determine a quality assurance result related to at least one of a target plan or a medical device based on the actual test result.

In some embodiments, the processing device 120 may be a single server or a server group. The server group may be centralized or distributed. In some embodiments, the processing device 120 may be local or remote. For example, the processing device 120 may access information and/or data from the medical device 110, the storage device 130, and/or the terminal device 140 via the network 150. As another example, the processing device 120 may be directly connected to the medical device 110, the terminal device 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal device 140. In some embodiments, the processing device 120 may be part of the medical device 110.

The storage device 130 may store data, instructions, and/or any other information. In some embodiments, the storage device 130 may store data obtained from the medical device 110, the processing device 120, and/or the terminal device 140. The data may include image data acquired by the medical device 110, algorithms and/or models for processing the image data, etc. For example, the storage device 130 may store a state of a medical device, a target plan of a target subject, a data set, or the like, or any combination thereof. As another example, the storage device 130 may store a prediction result, an actual test result, and/or a quality assurance result determined by the processing device 120. In some embodiments, the storage device 130 may store data and/or instructions that the processing device 120 and/or the terminal device 140 may execute or use to perform exemplary methods described in the present disclosure.

In some embodiments, the storage device 130 may include a mass storage, removable storage, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memories may include a random-access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), a high-speed RAM, etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components (e.g., the processing device 120, the terminal device 140) of the medical system 100. One or more components of the medical system 100 may access the data or instructions stored in the storage device 130 via the network 150. In some embodiments, the storage device 130 may be integrated into the medical device 110 and/or the processing device 120.

The terminal device 140 may be connected to and/or communicate with the medical device 110, the processing device 120, and/or the storage device 130. In some embodiments, the terminal device 140 may include a mobile device 141, a tablet computer 142, a laptop computer 143, or the like, or any combination thereof. In some embodiments, the terminal device 140 may include an input device, an output device, etc. The input device may include alphanumeric and other keys that may be input via a keyboard, a touchscreen (e.g., with haptics or tactile feedback), a speech input, an eye tracking input, a brain monitoring system, or any other comparable input mechanism. Other types of the input device may include a cursor control device, such as a mouse, a trackball, or cursor direction keys, etc. The output device may include a display, a printer, or the like, or any combination thereof.

The network 150 may include any suitable network that can facilitate the exchange of information and/or data for the medical system 100. In some embodiments, one or more components (e.g., the medical device 110, the processing device 120, the storage device 130, the terminal device 140) of the medical system 100 may communicate information and/or data with one or more other components of the medical system 100 via the network 150. For example, the processing device 120 and/or the terminal device 140 may obtain information stored in the storage device 130 via the network 150. In some embodiments, the network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (VPN), a satellite network, a telephone network, routers, hubs, witches, server computers, and/or any combination thereof.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart the scope of the present disclosure.

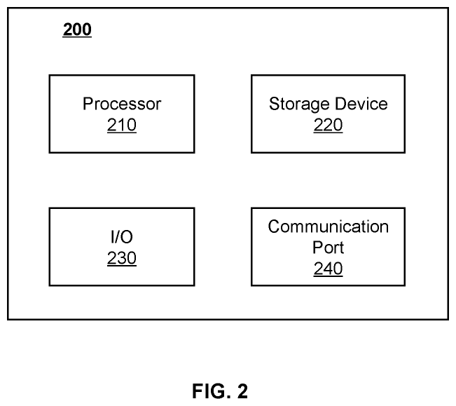

FIG. 2 is a schematic diagram illustrating exemplary hardware and/or software components of an exemplary computing device according to some embodiments of the present disclosure. In some embodiments, the processing device 120 and/or the terminal device 140 may be implemented on the computing device 200. As illustrated in FIG. 2, the computing device 200 may include a processor 210, a storage device 220, an input/output (I/O) 230, and a communication port 240.

The processor 210 may execute computer instructions (e.g., program code) and perform functions of the processing device 120 in accordance with techniques described herein. The computer instructions may include, for example, routines, programs, objects, components, data structures, procedures, modules, and functions, which perform particular functions described herein. For example, the processor 210 may process image data obtained from the medical device 110, the terminal device 140, the storage device 130, and/or any other component of the medical system 100. In some embodiments, the processor 210 may include one or more hardware processors, such as a microcontroller, a microprocessor, a reduced instruction set computer (RISC), an application specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field programmable gate array (FPGA), an advanced RISC machine (ARM), a programmable logic device (PLD), any circuit or processor capable of executing one or more functions, or the like, or any combination thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both process A and process B, it should be understood that process A and process B may also be performed by two or more different processors jointly or separately in the computing device 200 (e.g., a first processor executes process A and a second processor executes process B, or the first and second processors jointly execute processes A and B).

The storage device 220 may store data/information obtained from the medical device 110, the terminal device 140, the storage device 130, and/or any other component of the medical system 100. The storage device 220 may be similar to the storage device 130 described in connection with FIG. 1, and the detailed descriptions are not repeated here.

The I/O 230 may input and/or output signals, data, information, etc. In some embodiments, the I/O 230 may enable a user interaction with the processing device 120. In some embodiments, the I/O 230 may include an input device and an output device. Examples of the input device may include a keyboard, a mouse, a touchscreen, a microphone, a sound recording device, or the like, or a combination thereof. Examples of the output device may include a display device, a loudspeaker, a printer, a projector, or the like, or a combination thereof. Examples of the display device may include a liquid crystal display (LCD), a light-emitting diode (LED)-based display, a flat panel display, a curved screen, a television device, a cathode ray tube (CRT), a touchscreen, or the like, or a combination thereof.

The communication port 240 may be connected to a network (e.g., the network 150) to facilitate data communications. The communication port 240 may establish connections between the processing device 120 and the medical device 110, the terminal device 140, and/or the storage device 130. The connection may be a wired connection, a wireless connection, any other communication connection that can enable data transmission and/or reception, and/or any combination of these connections. The wired connection may include, for example, an electrical cable, an optical cable, a telephone wire, or the like, or any combination thereof. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. In some embodiments, the communication port 240 may be and/or include a standardized communication port, such as RS232, RS485. In some embodiments, the communication port 240 may be a specially designed communication port. For example, the communication port 240 may be designed in accordance with the digital imaging and communications in medicine (DICOM) protocol.

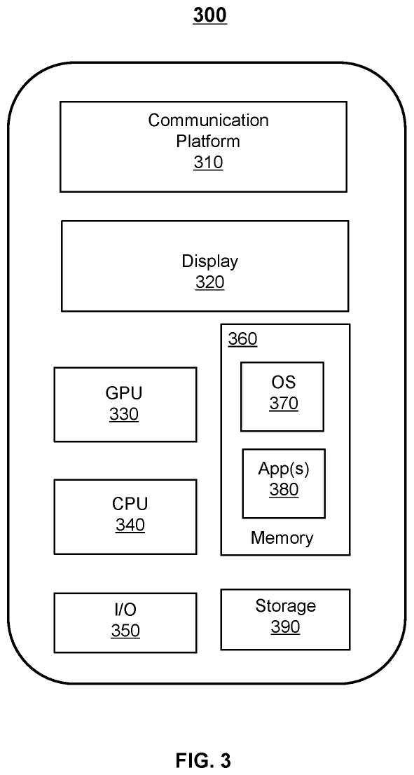

FIG. 3 is a schematic diagram illustrating exemplary hardware and/or software components of an exemplary mobile device according to some embodiments of the present disclosure. In some embodiments, the terminal device 140 and/or the processing device 120 may be implemented on a mobile device 300, respectively.

As illustrated in FIG. 3, the mobile device 300 may include a communication platform 310, a display 320, a graphics processing unit (GPU) 330, a central processing unit (CPU) 340, an I/O 350, a memory 360, and storage 390. In some embodiments, any other suitable component, including but not limited to a system bus or a controller (not shown), may also be included in the mobile device 300.

In some embodiments, the communication platform 310 may be configured to establish a connection between the mobile device 300 and other components of the medical system 100, and enable data and/or signal to be transmitted between the mobile device 300 and other components of the medical system 100. For example, the communication platform 310 may establish a wireless connection between the mobile device 300 and the medical device 110, and/or the processing device 120. The wireless connection may include, for example, a Bluetooth™ link, a Wi-Fi™ link, a WiMax™ link, a WLAN link, a ZigBee link, a mobile network link (e.g., 3G, 4G, 5G), or the like, or any combination thereof. The communication platform 310 may also enable the data and/or signal between the mobile device 300 and other components of the medical system 100. For example, the communication platform 310 may transmit data and/or signals inputted by a user to other components of the medical system 100. The inputted data and/or signals may include a user instruction. As another example, the communication platform 310 may receive data and/or signals transmitted from the processing device 120. The received data and/or signals may include imaging data acquired by the medical device 110.

In some embodiments, a mobile operating system (OS) 370 (e.g., iOS™ Android™, Windows Phone™) and one or more applications (App(s)) 380 may be loaded into the memory 360 from the storage 390 in order to be executed by the CPU 340. The applications 380 may include a browser or any other suitable mobile apps for receiving and rendering information from the processing device 120. User interactions with the information stream may be achieved via the I/O 350 and provided to the processing device 120 and/or other components of the medical system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or another type of work station or terminal device, although a computer may also act as a server if appropriately programmed. It is believed that those skilled in the art are familiar with the structure, programming and general operation of such computer equipment and as a result the drawings should be self-explanatory.

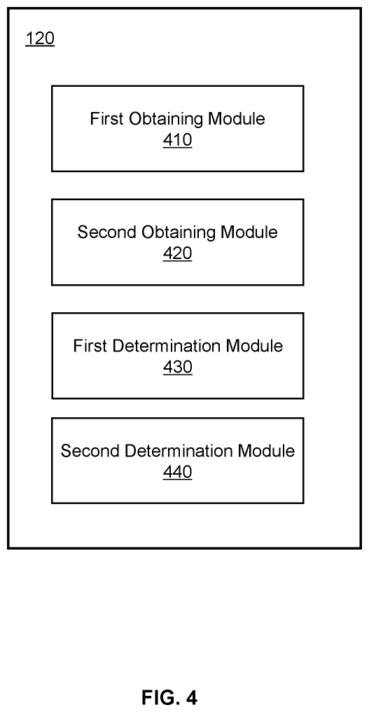

FIG. 4 is a schematic diagram illustrating an exemplary processing device according to some embodiments of the present disclosure. In some embodiments, the processing device 120 may include a first obtaining module 410, a second obtaining module 420, a first determination module 430, and a second determination module 440.

In some embodiments, the first obtaining module 410 may be configured to obtain a state of a medical device. The second obtaining module 420 may be configured to obtain a target plan of a target subject. The first determination module 430 may be configured to determine a prediction result based on the state of the medical device and the target plan of the target subject. The second determination module 440 may be configured to determine whether a quality assurance test passes based on the prediction result. In response to determining that the quality assurance test passes, the second determination module 440 may be configured to control the medical device to perform a medical operation on the target subject based on the target plan. In response to determining that the quality assurance test does not pass, the second determination module 440 may be configured to adjust the target plan, or determine a reason that the quality assurance test does not pass based on the state of the medical device, the target plan of the target subject, and the prediction result.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the first obtaining module 410 and the second obtaining module 420 may be combined into a single module. As another example, the first determination module 430 and the second determination module 440 may be combined into a single module. In some embodiments, one or more modules may be added or omitted in the processing device 120. For example, the processing device 120 may further include a storage module (not shown in FIG. 4) configured to store data and/or information (e.g., a state of a medical device, a target plan of a target subject, a prediction result) associated with the medical system 100. As another example, the processing device 120 may further include a training module (not shown in FIG. 4) configured to train a model (e.g., a first model and/or a second model described elsewhere in the present disclosure).

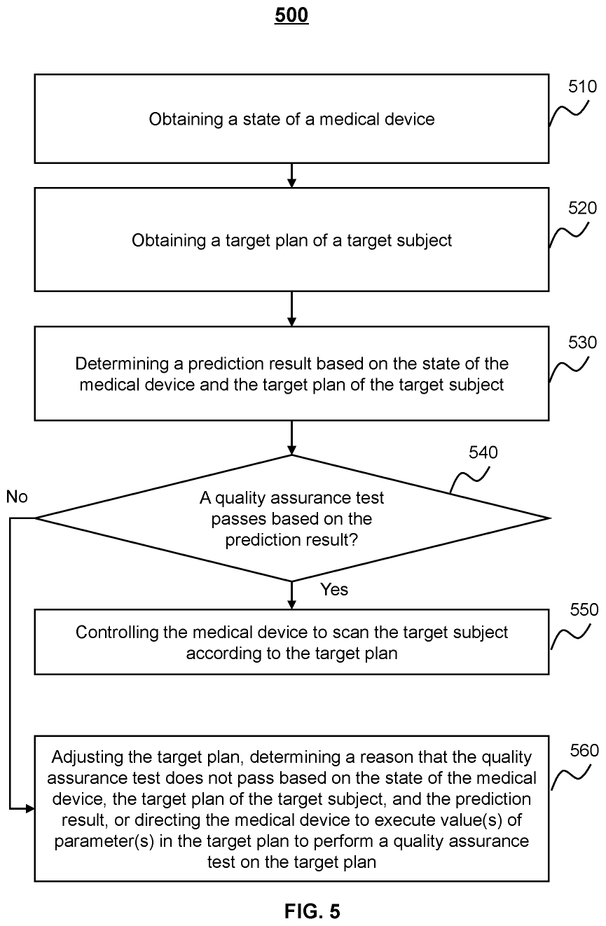

FIG. 5 is a flowchart illustrating an exemplary process for determining whether a quality assurance test passes according to some embodiments of the present disclosure. In some embodiments, process 500 may be executed by the medical system 100. For example, the process 500 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 500. The operations of the illustrated process presented below are intended to be illustrative. In some embodiments, the process 500 may be accomplished with one or more additional operations not described and/or without one or more of the operations discussed. Additionally, the order of the operations of process 500 illustrated in FIG. 5 and described below is not intended to be limiting.

In 510, the processing device 120 (e.g., the first obtaining module 410) may obtain a state of a medical device.

In some embodiments, the processing device 120 may obtain state information of the medical device. The state information of the medical device may reflect the state of the medical device. For example, the state information of the medical device may include one or more operation parameters of the medical device. In some embodiments, the state of the medical device may be predicted by directing the medical device to execute a plan (e.g., a radiotherapy plan).

In some embodiments, the state of the medical device may include beam and dose information (e.g., beam information corresponding to a plurality of dose rates), positioning accuracy information of at least one component (e.g., a gantry, a radiation source, a collimator) of the medical device, operation error information of the at least one component of the medical device, or the like, or any combination thereof.

The beam and dose information may include a beam flatness, a beam symmetry, a beam linearity, a beam output correction, a beam output consistency, or the like, or any combination thereof. The beam output correction may be a monitor unit (MU) correction. For example, after the beam output correction is performed on the medical device, a central dose at the maximum dose depth may be calibrated as 1MU=1cGy under a radiation field of 10 cm×10 cm. In some embodiments, the beam and dose information may include beam information (e.g., the beam flatness, the beam symmetry, the beam linearity, the beam output consistency) corresponding to a plurality of dose rates. For example, the beam and dose information may include a relationship between dose rate and beam flatness, a relationship between dose rate and beam symmetry, a relationship between dose rate and beam linearity, a relationship between dose rate and beam output consistency, or the like, or any combination thereof. The plurality of dose rates may be set by a user (e.g., a doctor, a technician) of the medical system 100, or by one or more components (e.g., the processing device 120) of the medical system 100 according to different situations.

The positioning accuracy information of at least one component of the medical device may include a positioning accuracy of a gantry rotation isocenter, a positioning accuracy of a collimator rotation isocenter, a positioning accuracy of a scanning table, a positioning accuracy of a collimator (e.g., a multi-leaf collimator), or the like, or any combination thereof. The positioning accuracy of the collimator may include a positioning accuracy of a leaf of a multi-leaf collimator, an accuracy of a movement speed of the leaf of the multi-leaf collimator, a positioning repeatability of the leaf of the multi-leaf collimator, or the like, or any combination thereof.

In some embodiments, the positioning accuracy information of the at least one component of the medical device may include positioning accuracy information of the at least one component of the medical device corresponding to a plurality of locations. The plurality of locations may include a plurality of gantry angles, a plurality of collimator positions (e.g., a position of a primary collimator, a position of a secondary collimator), a plurality of collimator angles (e.g., an angle of a primary collimator, an angle of a secondary collimator), or the like, or any combination thereof. For example, the positioning accuracy information of the collimator may include the positioning accuracy information of the collimator corresponding to a plurality of gantry angles. The plurality of locations may be set by a user (e.g., a doctor, a technician) of the medical system 100, or by one or more components (e.g., the processing device 120) of the medical system 100 according to different situations. For example, the plurality of gantry angles may be set as 30°, 60°, 120°, 180°, 240°, 300°, etc.

The operation error information of the at least one component of the medical device may include a systematic error, a random error, or the like, or a combination thereof. As used herein, a systematic error of a component may refer to a deviation of the whole component. For example, a systematic error of a multi-leaf collimator may be an overall offset of all leaves in the multi-leaf collimator (e.g., all leaves in the multi-leaf collimator are offset by a certain distance in a certain direction as a whole). As used herein, a random error of a component may refer to a deviation of a sub-component in the component. For example, offsets of a plurality of leaves in the multi-leaf collimator may be different (e.g., offsets of the leaves of the multi-leaf collimator may usually satisfy a Gaussian distribution), and the random error of the multi-leaf collimator may be an offset of one leaf in the multi-leaf collimator (e.g., the leaf is offset by a certain distance in a certain direction).

In some embodiments, the operation error information of the at least one component of the medical device may include operation error information of the at least one component of the medical device corresponding to the plurality of locations (e.g., the plurality of gantry angles, the plurality of collimator positions, the plurality of collimator angles). For example, the operation error information of the at least one component of the medical device may include the systematic error (or the random error) of the multi-leaf collimator corresponding to the plurality of gantry angles, the systematic error (or the random error) of the multi-leaf collimator corresponding to the plurality of collimator positions, the systematic error (or the random error) of the multi-leaf collimator corresponding to the plurality of collimator angles, or the like, or any combination thereof.

Since the states of medical device under different conditions (e.g., different dose rates, different locations) are usually different, for example, the beam information corresponding to different dose rates, the positioning accuracy information of the at least component of the medical device corresponding to different locations, and/or the operation error information of the at least one component of the medical device corresponding to the plurality of locations are usually different, by fully considering the states of the medical device under different conditions in the quality assurance test, the quality assurance test may be accurate and effective.

In some embodiments, the processing device 120 may determine the operation error information of the at least one component of the medical device based on operation information (e.g., historical operation information) of the medical device.

In some embodiments, the processing device 120 may obtain the operation information (e.g., the historical operation information) of the medical device. The operation information of the medical device may include a position, a movement speed, a movement acceleration, a movement distance, a movement direction, or the like, or any combination thereof, of the at least one component of the medical device. In some embodiments, the operation information of the medical device may be stored in a storage device (e.g., the storage device 130) of the medical system 100 or an external storage device. The processing device 120 may retrieve the operation information from the storage device of the medical system 100 or the external storage device. In some embodiments, processing device 120 may obtain historical quality assurance test data associated with the medical device and obtain the operation information of the medical device based on the historical quality assurance test data. The historical quality assurance test may include daily, weekly, monthly, and/or annual quality assurance tests for the medical device. The historical quality assurance test data may include a historical log file, a historical EPID image, or the like, or any combination thereof.

Further, the processing device 120 may determine the operation error information of the at least one component of the medical device based on the operation information of the medical device. In some embodiments, the processing device 120 may obtain actual operation information (e.g., actual historical operation information) and expected operation information (e.g., expected historical operation information) of the at least one component of the medical device based on the operation information (e.g., the historical operation information) of the medical device. The expected operation information (e.g., an expected position, an expected movement speed, an expected movement acceleration, an expected movement distance, an expected movement direction) of the at least one component may be set by a user (e.g., a doctor, a technician) of the medical system 100 via a control device (e.g., the terminal device 140) of the medical device. For example, the processing device 120 may obtain the actual operation information and the expected operation information of the at least one component from the historical log file of the medical device. As another example, a detection device (e.g., a position encoder, a speed sensor, an acceleration sensor) may be installed on the at least one component of the medical device to detect the actual operation information (e.g., an actual position, an actual movement speed, an actual movement acceleration, an actual movement distance, an actual movement direction) of the at least one component. The processing device 120 may then determine the operation error information of the at least one component of the medical device based on the actual operation information and the expected operation information of the at least one component of the medical device. For example, a user (e.g., a doctor) of the medical device 100 may determine an expected rotation angle of a gantry of the medical device is 10° according to a radiotherapy plan, and the expected rotation angle of the gantry of 10° may be input into the control device of the medical device to control the rotation of the gantry. Due to the operation error of the gantry, an actual rotation angle of the gantry may be measured as 12°, and the processing device 120 may determine that the operation error of the gantry is 2° (12°−10°=2°).

In some embodiments, the processing device 120 may determine the operation error information of the at least one component of the medical device based on the operation information of the medical device using a first error model. The first error model refers to an algorithm or process configured to determine the operation error information of the at least one component of the medical device based on the operation information of the medical device. For example, the operation information of the medical device may be input into the first error model, and the first error model may output the operation error information of the at least one component of the medical device.

In some embodiments, the processing device 120 may determine the operation error information of the at least one component of the medical device based on a simulated image (and/or a test image) and a radiotherapy plan using a second error model. The simulated image may be an EPID image determined based on the radiotherapy plan according to a data simulation algorithm. The test image may be an EPID image obtained using an imaging device (e.g., an EPID) by directing the medical device to execute the radiotherapy plan. For example, the simulation image (and/or the test image) and the radiotherapy plan may be input into the second error model, and the second error model may output the operation error information of the at least one component of the medical device.

In some embodiments, the first error model and/or the second error model may be a machine learning model. For example, the first error model and/or the second error model may be constructed based on a convolutional neural network (CNN), a fully convolutional neural network (FCN), a generative adversarial network (GAN), a U-shape network (UNet), a residual network (ResNet), a dense convolutional network (DenseNet), a deep stacking network, a deep belief network (DBN), a stacked auto-encoders (SAE), a logistic regression (LR) model, a support vector machine (SVM) model, a decision tree model, a naive Bayesian model, a random forest model, a restricted Boltzmann machine (RBM), a gradient boosting decision tree (GBDT) model, a LambdaMART model, an adaptive boosting model, a recurrent neural network (RNN) model, a hidden Markov model, a perceptron neural network model, a Hopfield network model, or the like, or any combination thereof.

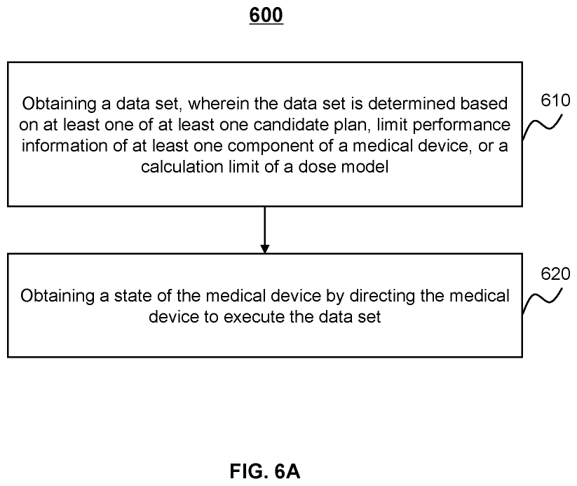

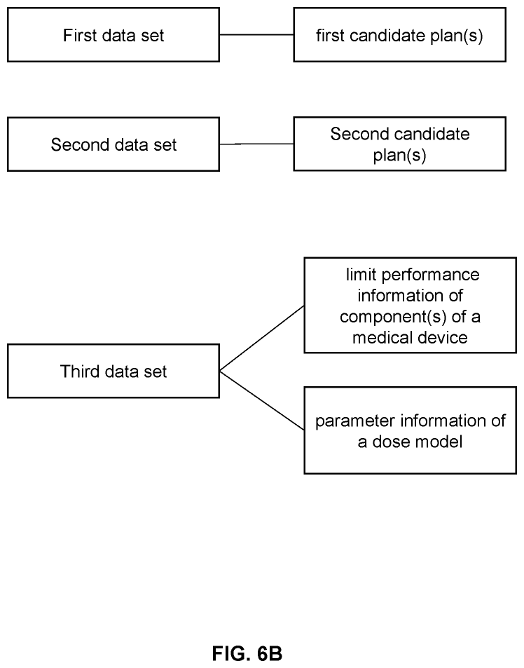

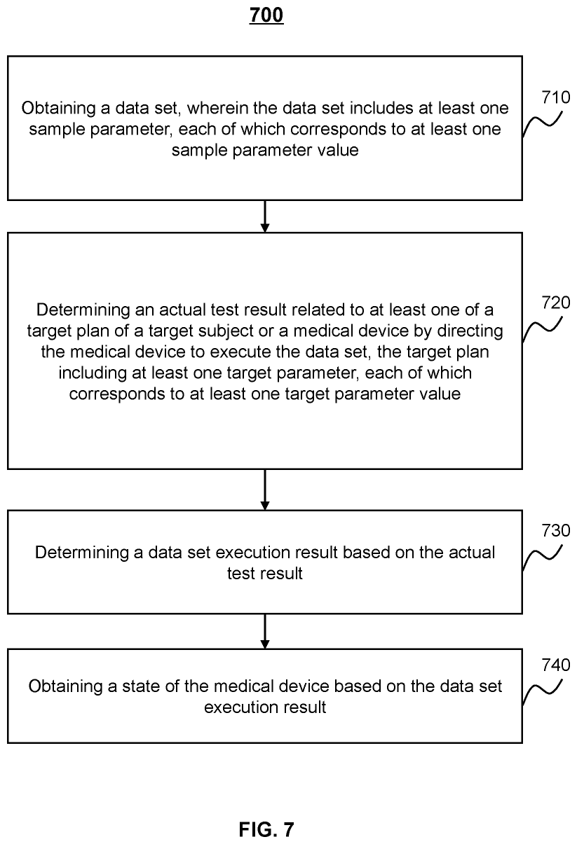

In some embodiments, the state of the medical device may also include a data set execution result. In some embodiments, the data set execution result may be obtained by directing the medical device to execute a data set. The data set execution result may reflect the state of the medical device. For example, the beam and dose information, the positioning accuracy information of the at least one component of the medical device, and/or the operation error information of the at least one component of the medical device may be estimated based on the data set execution result. More descriptions of the data set and the data set execution result may be found elsewhere in the present disclosure (e.g., FIGS. 6, 7, and descriptions thereof).

With the extension of the use time of medical device and the influence of the use environment, the state of medical device may change, and different states of medical device may have a great impact on a quality assurance test result. For example, for a same radiotherapy plan, when at least one of the beam and dose information, the positioning accuracy information of the at least one component of the medical device, or the operation error information of the at least one component of the medical device changes, prediction results (e.g., a predicted image of the target subject, a predicted gamma passing rate, a predicted dose distribution) may be different. Therefore, by fully considering the state of medical device in the quality assurance test, the quality assurance test may be accurate and effective.

In 520, the processing device 120 (e.g., the second obtaining module 420) may obtain a target plan of a target subject.

In some embodiments, the target subject may be a patient to be scanned (imaged or treated) using the medical device. The target plan (e.g., a target radiotherapy plan) may indicate how a radiotherapy operation is performed on the target subject, or more specifically, how one or more radiation beams are delivered to a target area of the target subject. In some embodiments, the target plan may include an intensity-modulated radiotherapy plan, a dynamic rotation intensity-modulated radiotherapy plan, an image-guided radiotherapy plan, a bio-guided radiotherapy plan, a dose-guided radiotherapy plan, an online adaptive radiotherapy plan, or the like, or any combination thereof.

In some embodiments, the target plan may include a radiotherapy parameter associated with a radiotherapy process of the target subject. The radiotherapy parameter may include a dose rate (e.g., MUs/min) of a radiation source (e.g., a treatment radiation source), a change rate of the dose rate, a radiation duration, a gantry angle (corresponding to a specific time period or time point), a gantry movement (e.g., rotation) speed (corresponding to a specific time period or time point), a gantry movement acceleration, a collimator angle (corresponding to a specific time period or time point), a collimator rotation speed (corresponding to a specific time period or time point), a parameter (e.g., a position, a movement speed, a movement acceleration, a movement direction) associated with a leaf of a multi-leaf collimator, a position of a scanning table, an angle of the scanning table, a number (or count) of radiation fields (or sub-fields), a shape of a radiation field (or sub-field), an area of the radiation field (or sub-field), a perimeter of the radiation field (or sub-field), an angle of the radiation field, or the like, or any combination thereof.

In some embodiments, the sub-field may be a field with a certain shape through which radiation rays can pass. A shape of a superposition of multiple sub-fields (also referred to as a radiation field) may match a shape of a target area of the target subject. A number (or count) of sub-fields may be determined based on a type of the medical device, a location of the target area, an area of the target area, a shape of the target area, etc. A shape and/or an area of the sub-field may be controlled based on positions of the leaves and the tungsten gate in the multi-leaf collimator. During a radiotherapy operation, positions, movement directions, and/or movement speeds of the leaves in the multi-leaf collimator may be controlled to form the sub-fields with different shapes and/or areas.

The dose rate of the radiation source may refer to an exposure dose per unit time. For example, the dose rate of the radiation source may be an intensity of a radiation beam generated by the radiation source passing through the radiation field (or the sub-field) and irradiating the target area per unit time. The radiation duration may refer to an irradiation duration of a radiation beam generated by the radiation source on the target area. In some embodiments, a dose distribution may be determined based on the dose rates at specific angles and the radiation duration. The angle of the radiation field may refer to an incident angle of a radiation beam generated by the radiation source irradiating the target area.

The gantry angle may be related to a position of a radiation source of the medical device with reference to an isocenter of the medical device. For example, the gantry angle may be an angle between a vertical direction and a direction of a beam axis of a radiation beam emitted from the radiation source of the medical device. Merely by way of example, the radiation source may be fixedly or flexibly attached to the gantry, when the gantry rotates around a gantry rotation axis in a circular path, the radiation source attached on the gantry may rotate along with the gantry, and the target subject located in a scanning table may be imaged and/or treated from a plurality of gantry angles.

In some embodiments, the processing device 120 may obtain a plan image of the target subject. The plan image may refer to an image that is used to determine the target plan. The plan image may include information related to the target area of the target subject. For example, the plan image may show the tumor as well as tissues and/or organs nearby the tumor in the target area. In some embodiments, the plan image may be a cone beam CT image, an MRI image, a PET-CT image, an MRI-CT image, or the like, or a combination thereof. In some embodiments, the plan image may be a historical medical image of the target subject. In some embodiments, the plan image may be the latest medical image obtained before the radiotherapy operation of the target subject. The processing device 120 may further determine the target plan based on the plan image of the target subject.

In some embodiments, the target plan of the target subject may be stored in a storage device (e.g., storage device 130) of the medical system 100 or an external storage device, and the processing device 120 may obtain the target plan from the storage device (e.g., storage device 130) or the external storage device.

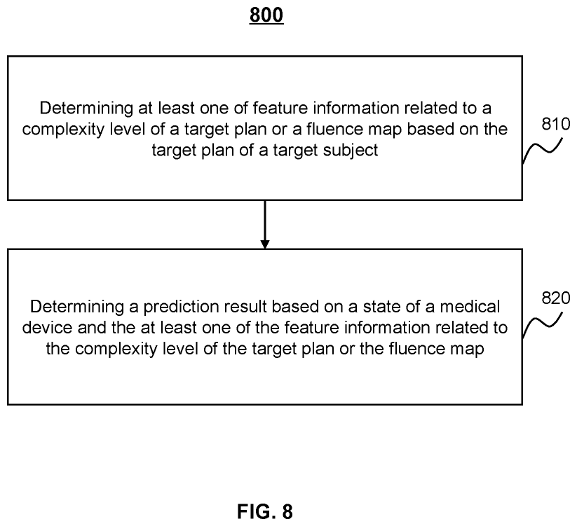

In some embodiments, the processing device 120 may further determine feature information related to a complexity level of the target plan and/or a target fluence map based on the target plan of the target subject. More descriptions of the feature information related to the complexity level of the target plan and the target fluence map may be found elsewhere in the present disclosure (e.g., FIG. 8 and descriptions thereof).

In 530, the processing device 120 (e.g., the first determination module 430) may determine a prediction result based on the state of the medical device and the target plan of the target subject.

In some embodiments, before operation 530, the processing device 120 may determine whether the state of the medical device satisfies a preset condition.

For example, the processing device 120 may determine whether the beam information (e.g., the beam flatness, the beam symmetry, the beam linearity, the beam output consistency) corresponding to the plurality of dose rates satisfies a corresponding condition (e.g., a flatness threshold, a symmetry threshold, a linearity threshold, an output consistency threshold). In response to determining that the beam information corresponding to the plurality of dose rates satisfies the corresponding condition (e.g., the beam flatness is higher than the flatness threshold, the beam symmetry is higher than the symmetry threshold, the beam linearity is higher than the linearity threshold, the beam output consistency is higher than the output consistency threshold), the processing device 120 may determine that the state of the medical device satisfies the preset condition.

As another example, the processing device 120 may determine whether the positioning accuracy information of the at least one component of the medical device is greater than a corresponding accuracy threshold. In response to determining that the positioning accuracy information of the at least one component of the medical device is greater than the corresponding accuracy threshold, the processing device 120 may determine that the state of the medical device satisfies the preset condition.

As still another example, the processing device 120 may determine whether the operation error information of the at least one component of the medical device is less than a corresponding error threshold. In response to determining that the operation error information of the at least one component of the medical device is less than the corresponding error threshold, the processing device 120 may determine that the state of the medical device satisfies the preset condition.

As still another example, the processing device 120 may determine whether the state of the medical device satisfies the preset condition based on the data set execution result. More descriptions for determining whether the state of the medical device satisfies the preset condition based on the data set execution result may be found elsewhere in the present disclosure (e.g., FIG. 6A and descriptions thereof).

In some embodiments, the preset condition (e.g., the flatness threshold, the symmetry threshold, the linearity threshold, the output consistency threshold, the accuracy threshold, the error threshold) may be manually set by a user (e.g., a doctor, a technician) of the medical system 100, or automatically set by one or more components (e.g., the processing device 120) of the medical system 100 according to different situations. For example, the preset condition may include a recommended value or a default value determined by the medical system 100.

In some embodiments, since the change degrees of different types of state of the medical device over time may be different, the checking frequencies of different types of state of the medical device may be different. For example, for a parameter (e.g., an accelerator output, a positioning accuracy of a multi-leaf collimator) associated with the state of the medical device that is easy to change, the checking frequency may be relatively high (e.g., once a day, once a week), to ensure the accuracy of the radiotherapy process. As another example, for a parameter (e.g., a positioning accuracy of a gantry rotation, a positioning accuracy of an isocenter of the medical device, a positioning accuracy of a scanning table) associated with the state of the medical device that is not easy to change, the checking frequency may be relatively low (e.g., once a month, once a year), to reduce the time of the quality assurance test and improve the efficiency of the quality assurance test.

In response to determining that the state of the medical device satisfies the preset condition, the processing device 120 may determine the prediction result based on the state of the medical device and the target plan of the target subject. In some embodiments, the prediction result may include a predicted image (e.g., a predicted EPID image) of the target subject, a predicted gamma passing rate, a predicted dose distribution (e.g., a 2D dose distribution, a 3D dose distribution), or the like, or any combination thereof. In some embodiments, the prediction result may also include a predicted dose measurement result of a dose measurement device. The dose measurement device may include a dose film, a water tank, a rotating radiation dosimeter (ArcCheck), a multi-sequence plane dosimeter (MapCheck), etc. As used herein, a dose distribution may refer to a spatial distribution of energy deposition in a target subject irradiated by radioactive particles such as photons or charged particles. For example, the dose distribution may indicate the amount of radiation delivered to one or more portions of the target subject and/or an absorbed dose that is absorbed by the one or more portions of the target subject.

In some embodiments, the processing device 120 may determine the prediction result based on the state of the medical device and the target plan of the target subject using a first model. As used herein, a first model refers to an algorithm or process configured to determine a prediction result based on a state of a medical device and a target plan of a target subject. In some embodiments, the processing device 120 may input the state of the medical device, the target plan of the target subject (e.g., the feature information related to the complexity level of the target plan and/or the target fluence map determined based on the target plan as described elsewhere in the present disclosure (e.g., FIG. 7 and descriptions thereof)), parameter information of a dose model (e.g., a type of the dose model, a parameter of the dose model, as described elsewhere in the present disclosure (e.g., FIG. 6A and descriptions thereof)), a medical image of the target subject, or the like, or any combination thereof, into the first model, and the first model may output the predicted image of the target subject, the predicted gamma passing rate, the predicted dose distribution, or the like, or any combination thereof.

The medical image may include a CT image, a PET image, an MRI image, or the like, or any combination thereof. In some embodiments, the medical image may be a historical medical image obtained during a historical imaging or treatment process of the target subject. In some embodiments, the medical image may be the latest medical image obtained before (e.g., a few days ago, a few hours ago) the radiotherapy operation of the target subject, and may reflect a current state of the target subject. For example, the medical image may be the plan image used to determine the target plan. As another example, the medical image may be a positioning image of the target subject used to position the target subject before the radiotherapy operation. According to some embodiments of the present disclosure, by inputting the medical image of the target subject into the first model, the accuracy and rationality of the prediction result output by the first model can be improved, which can make the prediction result clinically meaningful.

In some embodiments, when the input of the first model includes the medical image of the target subject, the first model may output a predicted 3D dose distribution in a body of the target subject. For example, the processing device 120 may input the state of the medical device, the target plan of the target subject, and the medical image of the target subject into the first model, and the first model may output a predicted dose volume histogram of the target subject.

In some embodiments, when the output of the first model includes the predicted EPID image, the target fluence map of the target subject may need to be one of the inputs of the first model. For example, the processing device 120 may input the state of the medical device and the target fluence map of the target subject into the first model, and the first model may output the predicted EPID image. As another example, the processing device 120 may input the state of the medical device, the target fluence map of the target subject, and the feature information related to the complexity level of the target plan into the first model, and the first model may output the predicted EPID image. As still another example, the processing device 120 may input the state of the medical device, the target fluence map of the target subject, and the parameter information of the dose model into the first model, and the first model may output the predicted EPID image. As still another example, the processing device 120 may input the state of the medical device, the target fluence map of the target subject, the feature information related to the complexity level of the target plan, and the parameter information of the dose model into the first model, and the first model may output the predicted EPID image. As still another example, the processing device 120 may input the state of the medical device, and the target fluence map of the target subject (and/or the feature information related to the complexity level of the target plan) into the first model, and the first model may output the predicted gamma passing rate. As still another example, the processing device 120 may input the state of the medical device, the target fluence map of the target subject (and/or the feature information related to the complexity level of the target plan), and the parameter information of the dose model into the first model, and the first model may output the predicted gamma passing rate.

In some embodiments, since the target fluence map can reflect information related to radiotherapy parameters (e.g., parameters related to a radiation field and/or a sub-field) and parameter information of the dose model to a certain extent, when the target fluence map is used as the input of the first model, the information related to the radiotherapy parameters and the parameter information of the dose model may not need to be used as the input of the first model.

In some embodiments, the processing device 120 may determine the prediction result based on the state of the medical device and the target plan of the target subject according to one or more analytical algorithms. For example, the state of the medical device, the target plan of the target subject, the parameter information of the dose model, the medical image of the target subject, or the like, or any combination thereof, may be used as independent variables in an empirical formula of dose determination, and the prediction result may be used as a dependent variable in the empirical formula of dose determination. In some embodiments. The empirical formula of dose determination may be determined based on historical radiotherapy data and/or simulated experimental data.

In 540, the processing device 120 (e.g., the second determination module 440) may determine whether a quality assurance test passes based on the prediction result.

For example, the processing device 120 may determine whether the quality assurance test passes based on the predicted gamma passing rate and a first threshold. In response to determining that the predicted gamma passing rate is not greater than the first threshold, the processing device 120 may determine that the quality assurance test does not pass. In response to determining that the predicted gamma passing rate is greater than the first threshold, the processing device 120 may determine that the quality assurance test passes.

As another example, the processing device 120 may determine whether the quality assurance test passes based on a difference between the predicted image and a calculated image, and a second threshold. The calculated image (e.g., a calculated EPID image) may be an EPID image determined by the medical system 100 (e.g., a treatment planning system (TPS) in the medical system 100) based on the target plan of the target subject. In response to determining that the difference between the predicted image and the calculated image is greater than the second threshold, the processing device 120 may determine that the quality assurance test does not pass. In response to determining that the difference between the predicted image and the calculated image is not greater than the second threshold, the processing device 120 may determine that the quality assurance test passes.

In some embodiments, the target subject may include a plurality of regions of interest (ROIs). The plurality of ROIs may include a planning target volume (PTV), an organ at risk (OAR), or other organs or tissues of the target subject. The prediction result may include predicted dose distributions (e.g., a dose volume histogram) corresponding to the plurality of ROIs respectively. In some embodiments, the processing device 120 may determine a weight corresponding to each ROI of the plurality of ROIs. In some embodiments, a weight corresponding to a specific ROI may reflect an importance of a dose difference between a planned dose distribution of the specific ROI and a predicted dose distribution of the specific ROI in determining whether the quality assurance test passes. For example, the higher the weight corresponding to the ROI is, the higher the importance of the dose difference between the planned dose distribution of the ROI and the predicted dose distribution of the ROI in determining whether the quality assurance test passes may be. In some embodiments, the weights corresponding to the OAR and the PTV may be set relatively high to ensure that tumor tissue receives a planning radiation dose and radiation dose that normal tissue receives is within a safe range.

Further, the processing device 120 may determine whether the quality assurance test passes based on the weights and the predicted dose distributions corresponding to the plurality of ROIs respectively. For example, for each ROI of the plurality of ROIs, the processing device 120 may determine whether a product of the dose difference between the planned dose distribution and the predicted dose distribution of the ROI, and the weight corresponding to the ROI is greater than a corresponding dose difference threshold. In response to determining that the product of the dose difference between the planned dose distribution and the predicted dose distribution of the ROI and the weight corresponding to the of the ROI is not greater than the corresponding dose difference threshold, the processing device 120 may determine that a test corresponding to the ROI passes.

In some embodiments, in response to determining that tests corresponding to all ROIs pass, the processing device 120 may determine that the quality assurance test passes. In some embodiments, in response to determining that a ratio of a number (or count) of passed tests corresponding to ROIs to a total number (or count) of tests corresponding to ROIs is greater than a ratio threshold (e.g., 90%, 95%, 99%), the processing device 120 may determine that the quality assurance test passes. In some embodiments, in response to determining that test(s) corresponding to ROI(s) with a relatively high weight (e.g., greater than a weight threshold) passes, the processing device 120 may determine that the quality assurance test passes.

In some embodiments, the first threshold, the second threshold, the dose difference threshold, and/or the ratio threshold may be manually set by a user (e.g., a doctor, a technician) of the medical system 100, or automatically set by one or more components (e.g., the processing device 120) of the medical system 100 according to different situations.

According to some embodiments of the present disclosure, by determining the predicted dose distribution of each ROI of the plurality of ROIs of the target subject, the difference between the predicted dose distribution and the planned dose distribution of each ROI of the target subject may be displayed comprehensively and intuitively, which can make the quality assurance test result have clinical significance. In addition, the weight and the dose difference threshold corresponding to the each ROI may be determined according to a feature of the each ROI (e.g., whether the ROI belongs to eye tissue, whether the ROI is the PTV or the OAR), which can improve the rationality and accuracy of the quality assurance test.

In 550, in response to determining that the quality assurance test passes, the processing device 120 (e.g., the second determination module 440) may control the medical device to perform a medical operation on the target subject based on the target plan.

For example, the processing device 120 may control the at least one component of the medical device to perform a radiotherapy operation on the target subject based on value(s) of radiotherapy parameter(s) in the target plan of the target subject.

In some embodiments, a user of (e.g., a doctor, a technician) of the medical system 100 may adjust or confirm the target plan based on the prediction result. The processing device 120 may control the at least one component of the medical device to perform the radiotherapy operation on the target subject based on value(s) of radiotherapy parameter(s) in an adjusted target plan or a confirmed target plan of the target subject.

In 560, in response to determining that the quality assurance test does not pass, the processing device 120 (e.g., the second determination module 440) may adjust the target plan, determine a reason that the quality assurance test does not pass based on the state of the medical device, the target plan of the target subject, and the prediction result, or direct the medical device to execute value(s) of parameter(s) in the target plan to perform a quality assurance test on the target plan.

In some embodiments, in response to determining that the quality assurance test does not pass, the processing device 120 may adjust value(s) of parameter(s) in the target plan. For example, the processing device 120 may adjust value(s) of parameter(s) in the target plan to adjust (e.g., decrease) a complexity level of the target plan.

In some embodiments, in response to determining that the quality assurance test does not pass, a quality assurance test may be performed on the target plan by directing the medical device to execute value(s) of parameter(s) in the target plan. In response to determining that the quality assurance test does not pass, the processing device 120 may adjust value(s) of parameter(s) in the target plan until the quality assurance test passes. In response to determining that the quality assurance test passes, the processing device may control the medical device to perform the medical operation on the target subject based on the target plan.

In some embodiments, in response to determining that the quality assurance test does not pass, the processing device 120 may determine a reason that the quality assurance test does not pass based on the state of the medical device, the target plan of the target subject, and the prediction result using a second model. As used herein, a second model refers to an algorithm or process configured to determine a reason that the quality assurance test does not pass based on a state of a medical device, a target plan of a target subject, parameter information of a dose model, a medical image of the target subject, a prediction result, or the like, or any combination thereof. For example, the processing device 120 may input a predicted EPID image (or a calculated EPID image), the state of the medical device, and the target plan of the target subject (e.g., the feature information related to the complexity level of the target plan and/or the target fluence map determined based on the target plan) into the second model, and the second model may output the reason that the quality assurance test does not pass. As another example, the processing device 120 may input the predicted EPID image (or the calculated EPID image), the state of the medical device, the target plan of the target subject (e.g., the feature information related to the complexity level of the target plan and/or the target fluence map determined based on the target plan), and the parameter information of the dose model into the second model, and the second model may output the reason that the quality assurance test does not pass.

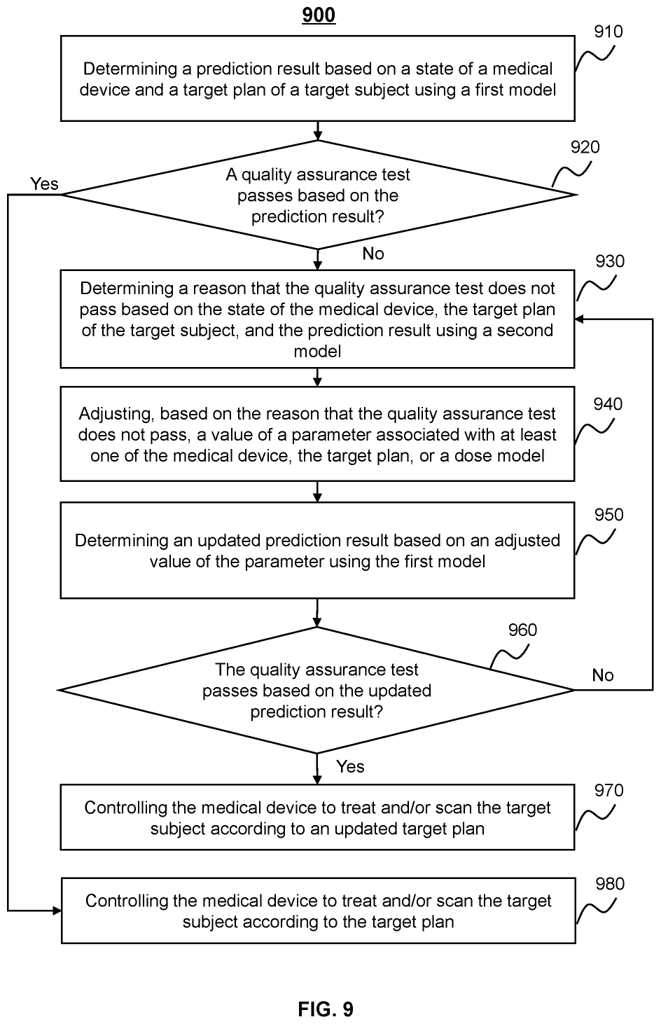

Further, the processing device 120 may adjust, based on the reason that the quality assurance test does not pass, a value of a parameter associated with at least one of the medical device, the target plan, or the dose model. The processing device 120 may determine an updated prediction result based on an adjusted value of the parameter using the first model. Then the processing device 120 may determine whether the quality assurance test passes based on the updated prediction result. In response to determining that the quality assurance test passes, the processing device 120 may control the medical device to treat and/or scan the target subject according to an updated target plan. The updated target plan may be determined based on the adjusted value of the parameter. More descriptions for adjusting the value of the parameter associated with the at least one of the medical device, the target plan, or the dose model based on the reason that the quality assurance test does not pass may be found elsewhere in the present disclosure (e.g., FIG. 9, and descriptions thereof).

In some embodiments, the first model and/or the second model may be a machine learning model. For example, the first model and/or the second model may be constructed based on a convolutional neural network (CNN), a fully convolutional neural network (FCN), a generative adversarial network (GAN), a U-shape network (UNet), a residual network (ResNet), a dense convolutional network (DenseNet), a deep stacking network, a deep belief network (DBN), a stacked auto-encoders (SAE), a logistic regression (LR) model, a support vector machine (SVM) model, a decision tree model, a naive Bayesian model, a random forest model, a restricted Boltzmann machine (RBM), a gradient boosting decision tree (GBDT) model, a LambdaMART model, an adaptive boosting model, a recurrent neural network (RNN) model, a hidden Markov model, a perceptron neural network model, a Hopfield network model, or the like, or any combination thereof. In the present disclosure, the first model and/or the second model may also be referred to as a quality assurance model.

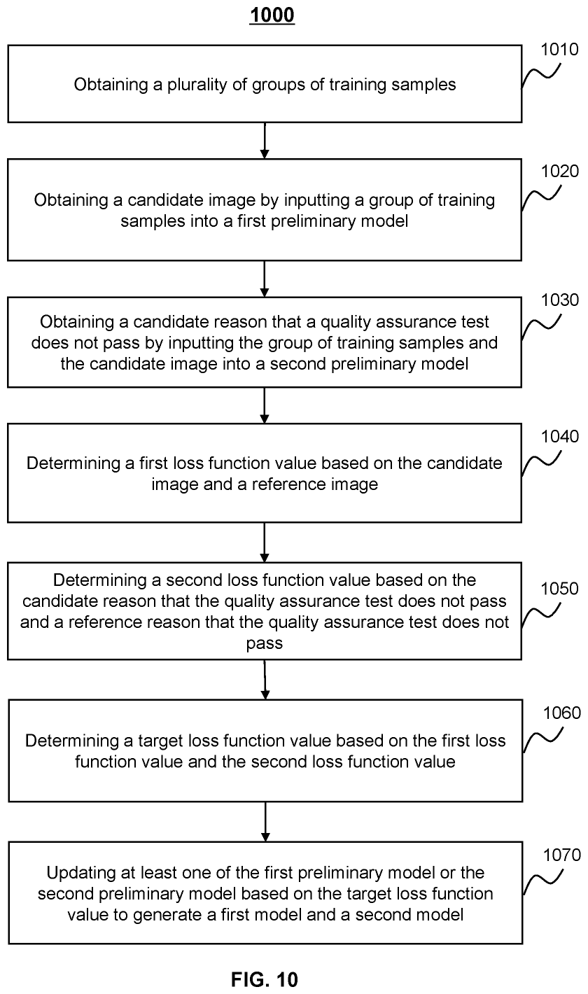

In some embodiments, the first model (and/or the second model) may be determined by training a first preliminary model (and/or a second preliminary model) using a plurality of groups of training samples. In some embodiments, the processing device 120 may train the first preliminary model (and/or the second preliminary model) to generate the first model (and/or the second model) according to a machine learning algorithm. The machine learning algorithm may include an artificial neural network algorithm, a deep learning algorithm, a decision tree algorithm, an association rule algorithm, an inductive logic programming algorithm, a support vector machine algorithm, a clustering algorithm, a Bayesian network algorithm, a reinforcement learning algorithm, a representation learning algorithm, a similarity and metric learning algorithm, a sparse dictionary learning algorithm, a genetic algorithm, a rule-based machine learning algorithm, or the like, or any combination thereof. The machine learning algorithm used to generate the first model (and/or the second model) may be a supervised learning algorithm, a semi-supervised learning algorithm, an unsupervised learning algorithm, or the like. More descriptions for obtaining the first model and the second model may be found elsewhere in the present disclosure (e.g., FIG. 10, and descriptions thereof).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, two or more operations may be combined into one operation. For example, operation 510 and operation 520 may be combined into one operation. In some embodiments, one or more operations may be added in process 500. For example, an operation for obtaining the parameter information of the dose model may be added in process 500. Accordingly, in operation 530, the processing device 120 may determine the prediction result based on the state of the medical device, the target plan of the target subject, and the parameter information of the dose model. As another example, an operation for obtaining the medical image of the target subject may be added in process 500. Accordingly, in operation 530, the processing device 120 may determine the prediction result based on the state of the medical device, the target plan of the target subject, the parameter information of the dose model, and the medical image of the target subject. As still another example, an operation for determine whether the state of the medical device satisfies the preset condition may be added before operation 530 in process 500. In response to determining that the state of the medical device satisfies the preset condition, the processing device 120 may determine the prediction result based on the state of the medical device and the target plan of the target subject in operation 530.

In some embodiments, operation 510, operation 520, and the operation for determining whether the state of the medical device satisfies the preset condition may be performed according to a preset frequency (e.g., daily, weekly, monthly, annually). Before the medical operation is performed on the target subject, operations 530 and 540 may be performed to determine whether the quality assurance test passes based on the prediction result. In some embodiments, operations 510-560 may be performed before the medical operation is performed on the target subject.

FIG. 6A is a flowchart illustrating an exemplary process for obtaining a state of a medical device according to some embodiments of the present disclosure. In some embodiments, process 600 may be executed by the medical system 100. For example, the process 600 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 600. The operations of the illustrated process presented below are intended to be illustrative. In some embodiments, the process 600 may be accomplished with one or more additional operations not described and/or without one or more of the operations discussed. Additionally, the order of the operations of process 600 illustrated in FIG. 6A and described below is not intended to be limiting.

In 610, the processing device 120 (e.g., the first obtaining module 410) may obtain a data set.

The data set may be a collection of data used for a quality assurance test of a medical device and/or a target plan of a target subject. In some embodiments, the data set may be determined based on at least one candidate plan, parameter information (e.g., limit performance information) of at least one component of the medical device, parameter information (e.g., a calculation limit) of a dose model, or the like, or any combination thereof. In some embodiments, the data set may include a clinical radiotherapy plan, a radiotherapy plan for determining the limit performance of the at least one component of the medical device, a radiotherapy plan for determining the calculation limit of the dose model, or the like, or any combination thereof.

In some embodiments, the data set may include a first data set, a second data set, a third data set, or the like, or any combination thereof.

In some embodiments, the processing device 120 may determine the first data set based on at least one first candidate plan (e.g., a historical radiotherapy plan of a candidate subject). A value of a first candidate parameter (e.g., a radiotherapy parameter in a radiotherapy process, as described in connection with operation 520 in FIG. 5) in the at least one first candidate plan may be within a first range.

In some embodiments, the first range may be set by a user (e.g., a doctor, a technician) based on user experience. In some embodiments, the first range may be determined based on value(s) of radiotherapy parameter(s) in a plurality of historical radiotherapy plans. For example, taking the radiotherapy parameter as the number (or count) of radiation fields as an example, if the number (or count) of radiation fields in most historical radiotherapy plans is between 1 and 5 (e.g., a ratio of radiotherapy plans with the number (or count) of radiation fields between 1 and 5 to a total number (or count) of radiotherapy plans is greater than a certain threshold (e.g., 90%, 95%, 99%)), the processing device 120 may determine that the first range is from 1 to 5. That is, the first data set may be considered as a set of data determined based on common clinical radiotherapy plans, and the range of values of the radiotherapy parameters in the first data set may be within the range of values of the corresponding radiotherapy parameter in the most historical radiotherapy plans.

In some embodiments, the processing device 120 may determine value(s) of radiotherapy parameter(s) in the first candidate plan as value(s) of corresponding radiotherapy parameter(s) in the first data set. In some embodiments, the processing device 120 may adjust the value(s) of the radiotherapy parameter(s) in the first candidate plan, and determine adjusted value(s) of the radiotherapy parameter(s) as the value(s) of the corresponding radiotherapy parameter(s) in the first data set.

In some embodiments, the processing device 120 may determine the first data set based on a plurality of first candidate plans using a parameter extraction model. The parameter extraction model refers to an algorithm or process configured to extract value(s) of radiotherapy parameter(s) in a plurality of candidate plans and/or determine a data set based on the plurality of candidate plans. For example, the processing device 120 may input the plurality of first candidate plans into the parameter extraction model. The parameter extraction model may extract value(s) of radiotherapy parameter(s) in the plurality of first candidate plan. The processing device 120 may determine extracted value(s) of the radiotherapy parameter(s) in the first candidate plan as the value(s) of the corresponding radiotherapy parameter(s) in the first data set. As another example, the processing device 120 may input the plurality of first candidate plans into the parameter extraction model. The parameter extraction model may output the first data set.

In some embodiments, the processing device 120 may determine the second data set based on at least one second candidate plan (e.g., a historical radiotherapy plan of a candidate subject). A value of a second candidate parameter (e.g., a radiotherapy parameter in a radiotherapy process, as described in connection with operation 520 in FIG. 5) in the at least one second candidate plan may be outside a second range. The second candidate plan may be a historical radiotherapy plan of the candidate subject.

The second range may be the same as or different from the first range. In some embodiments, the second range may be set by a user (e.g., a doctor, a technician) based on user experience. In some embodiments, the second range may be determined based on value(s) of radiotherapy parameter(s) in a plurality of historical radiotherapy plans. For example, taking the radiotherapy parameter as the number (or count) of radiation fields as an example, if the number (or count) of radiation fields in most historical radiotherapy plans is between 1 and 5 (e.g., a ratio of radiotherapy plans with the number (or count) of radiation fields between 1 and 5 to the total number of radiotherapy plans is greater than a certain threshold (e.g., 90%, 95%, 99%)), the processing device 120 may determine that the second range is greater than 5. As another example, the second range may be determined based on value(s) of radiotherapy parameter(s) in a special type of radiotherapy plan (e.g., a radiotherapy plan with a relatively large radiation field). As still another example, if value(s) of radiotherapy parameter(s) in a historical radiotherapy plan determined based on a special radiotherapy purpose is relatively high or relatively low (e.g., a relatively small area of a sub-field, a relatively high degree deviation of a sub-field from a beam center point), value(s) of radiotherapy parameter(s) in the historical radiotherapy plan may be determined as value(s) of corresponding radiotherapy parameter(s) in the second data set. That is, the second data set may be considered as a set of data determined based on uncommon clinical radiotherapy plans, and the range of values of the radiotherapy parameters in the second data set may be outside the range of values of the corresponding radiotherapy parameter in most historical radiotherapy plans.

The determination of the second data set may be similar to the determination of the first data set. For example, the processing device 120 may determine value(s) of radiotherapy parameter(s) in the second candidate plan as value(s) of corresponding radiotherapy parameter(s) in the second data set. As another example, the processing device 120 may determine the second data set based on a plurality of second candidate plans using the parameter extraction model.

In some embodiments, the processing device 120 may determine the third data set based on the limit performance information of the at least one component (e.g., a radiation source, a gantry, a scanning table, a leaf of a multi-leaf collimator) of the medical device, parameter information (e.g., the calculation limit) of the dose model, or the like, or any combination thereof. For example, the third data set may include value(s) of parameter(s) related to the limit performance of the at least one component of the medical device and/or value(s) of parameter(s) related to the calculation limit of the dose model.

The limit performance information of the at least one component may include the maximum movement speed of the at least one component, the maximum movement acceleration of the at least one component, a limit movement position of the at least one component, or the like, or any combination thereof. Since the performance of the at least one component of the medical device cannot exceed its limit performance, value(s) of radiotherapy parameter(s) in the third data set need to be reasonably determined according to the limit performance information of the at least one component of the medical device.

In some embodiments, the dose model may include an algorithm or a model that can simulate and/or determine a result (e.g., an EPID image, a dose distribution) related to does according to value(s) of radiotherapy parameter(s). For example, the dose model (or the dose algorithm) may include a Monte Carlo dose model, a pencil beam algorithm, a convolution algorithm, a machine learning algorithm, or the like, or any combination thereof. In some embodiments, the parameter information of the dose model may include a type of the dose model (e.g., a type of the dose algorithm), a parameter of the dose model (e.g., a parameter in the dose algorithm), or the like, or any combination thereof.

In some embodiments, the dose model may include a model of at least one component of the medical device. For example, the dose model may include an accelerator model (e.g., a beam model) used to determine an energy (e.g., an absorbed dose) deposited by a radiation beam in a target area and an organ at risk of a patient. The parameter information of the dose model may include a parameter of the model of the at least one component of the medical device. In some embodiments, the parameter of the accelerator model may include a parameter (e.g., a position parameter) of at least one component (e.g., a primary collimator, a flattening filter, a secondary collimator) of the accelerator. For example, the parameter of the accelerator model may include a deviation of a multi-leaf collimator, a transmittance of the multi-leaf collimator, a size of a leaf-tip structure of a leaf of the multi-leaf collimator, a width and a transmittance of a tongue-and-groove structure of the leaf of the multi-leaf collimator, or the like, or any combination thereof.

In some embodiments, the calculation limit of the dose model may reflect a calculation ability of the dose model. If the calculation ability of the dose model is relatively poor (e.g., a calculation accuracy is relatively low), a passing rate of the dose model in a calculation limit test may be relatively low. The calculation limit test may reflect the calculation ability of the dose model. In some embodiments, the calculation limit of the dose model may include the calculation accuracy of the dose model for a parameter value prone to calculation deviation. For example, since a radiation in a radiation field is usually non-uniformly distributed, a dose rate of the radiation near a boundary of the radiation field is relatively low, and the non-uniform distribution of the radiation may easily lead to errors in the dose calculation (especially the dose calculation at the boundary of the radiation field). Therefore, the accuracy of the dose model in calculating the dose at the boundary of the radiation field boundary may reflect the calculation ability of the dose model.

The use of the dose model may simplify the dose determination process and improve the efficiency of dose determination. However, the use of different types of dose models or different parameters of the dose model may have a great impact on a result of the quality assurance test. For example, for the same radiotherapy plan, when different parameters of the dose model are used, the predicted results may have significant differences. Therefore, by fully considering the parameter information of the dose model in the quality assurance test, the quality assurance test may be accurate and effective.

FIG. 6B is a schematic diagram illustrating an exemplary first data set, an exemplary second data set, and an exemplary third data set according to some embodiments of the present disclosure. As shown in FIG. 6B, the first data set may be determined based on at least one first candidate plan, the second data set may be determined based on at least one second candidate plan, and the third data set may be determined based on limit performance information of at least one component of a medical device and/or a calculation limit of a dose model.

In some embodiments, the data set may include other types of data sets. For example, a user (e.g., a doctor, a technician) of the medical system 100 may set value(s) of radiotherapy parameter(s) in a data set based on user experience. As another example, the user (e.g., the doctor, the technician) of the medical system 100 may set the a range of value(s) of radiotherapy parameter(s) in the data set, and the processing device 120 may randomly determine the value(s) of the radiotherapy parameter(s) in the data set based on the range of value(s) of the radiotherapy parameter(s) in the data set. As still another example, the processing device 120 may select a historical radiotherapy plan from a database randomly, and determine value(s) of radiotherapy parameter(s) in the selected historical radiotherapy plan as value(s) of corresponding radiotherapy parameter(s) in the data set.

In 620, the processing device 120 (e.g., the first obtaining module 410) may obtain a state of the medical device by directing the medical device to execute the data set.

In some embodiments, the processing device 120 may determine an actual test result by directing the medical device to execute the data set. The actual test result may include a test image (e.g., a radiation field image), a dose distribution (e.g., a measured dose distribution), or the like, or any combination thereof.

In some embodiments, the processing device 120 may determine the actual test result using a test device. In some embodiments, the test device may include an imaging device (e.g., an EPID). The actual test result may include a test image obtained using the imaging device by directing the medical device to execute the data set. For example, a radiotherapy operation may be performed based on value(s) of radiotherapy parameter(s) in the data set. The imaging device (e.g., the EPID) may receive the radiation from the radiation field to generate a radiation field image. The processing device 120 may determine the radiation field image as the actual test result. As another example, the processing device 120 may generate a measured dose distribution based on the radiation field image, and determine the measured dose distribution as the actual test result.

In some embodiments, the test device may include a third-party device (e.g., a phantom, a dose measurement device). The actual test result may include a dose distribution measured using the dose measurement device by directing the medical device to execute the data set. For example, the phantom may be placed on a scanning table, and a radiotherapy operation may be performed on the phantom based on the value(s) of the radiotherapy parameter(s) in the data set. The dose measurement device may measure a dose distribution of the phantom. The processing device 120 may determine the measured dose distribution as the actual test result. As another example, the radiotherapy operation may be performed on the phantom based on the value(s) of the radiotherapy parameter(s) in the data set. The imaging device (e.g., the EPID) may generate a radiation field image of the phantom. The processing device 120 may generate the measured dose distribution based on the radiation field image. The processing device 120 may determine the radiation field image of the phantom and/or the measured dose distribution as the actual test result.

In some embodiments, the processing device 120 may determine a plurality of actual test results by directing the medical device to execute a plurality of data sets. For example, when a gantry angle is between 0° to 30°, the value(s) of the radiotherapy parameter(s) in the first data set may be executed by the medical device to determine a first actual test result. When the gantry angle is between 30° to 60°, the value(s) of the radiotherapy parameter(s) in the second data set may be executed by the medical device to determine a second actual test result. When the gantry angle is between 60° to 120°, the value(s) of the radiotherapy parameter(s) in the third data set may be executed by the medical device to determine a third actual test result.

In some embodiments, the processing device 120 may determine a predicted test result based on the data set using a test model. The test model may include the dose model, a data simulation algorithm, or the like, or a combination thereof. The prediction test result may include an image (e.g., a simulated image), a dose distribution (e.g., a simulated dose distribution, a planned dose distribution), or the like, or any combination thereof. In some embodiments, the processing device 120 may obtain simulated scanning data based on the data set according to the data simulation algorithm. Then the processing device 120 may generate a simulated image based on the simulated scanning data. In some embodiments, the processing device 120 may determine a simulated dose distribution based on the data set using the dose model. The planned dose distribution may refer to an expected dose distribution. The planned dose distribution may be determined by a user (e.g., a doctor) of the medical system 100, or one or more components (e.g., the processing device 120) of the medical system 100 according to an actual situation.

Further, the processing device 120 may determine a data set execution result based on the predicted test result and the actual test result. In some embodiments, the processing device 120 may determine the data set execution based on a difference between the predicted test result and the actual test result. For example, the processing device 120 may determine a difference between the measured dose distribution and the simulated dose distribution (or the planned dose distribution) as the data set execution result.

In some embodiments, the data set execution result may reflect the state of the medical device. For example, the smaller the difference between the actual test result and the predicted test result is, the better the state of the medical device may be, and the higher the accuracy to execute a radiotherapy plan using the medical device may be. In some embodiments, data set execution results corresponding to different types of data set may reflect different types of states of the medical device. For example, the execution result of the first data set may reflect the accuracy to execute a common clinical radiotherapy plan using the medical device. The execution result of the second data set and/or the execution result of the third data set may reflect the accuracy to execute an uncommon clinical radiotherapy plan using the medical device.

In some embodiments, the processing device 120 may determine whether the state of the medical device satisfies a preset condition based on the data set execution result. For example, the processing device 120 may determine whether the state of the medical device satisfies the preset condition based on a difference between the test image obtained by the imaging device (e.g., the EPID) and the simulated image, and a first difference threshold. The difference between a first image (e.g., the test image) and a second image (e.g., the simulated image) may be determined based on a difference between an average gray value of pixels (or voxels) of the first image and an average gray value of pixels (or voxels) of the second image. In response to determining that the difference between the test image and the simulated image is greater than the first difference threshold, the processing device 120 may determine that the data set execution result does not satisfy an execution condition, and the state of the medical device does not satisfy the preset condition. In response to determining that the difference between the test image and the simulated image is not greater than the first difference threshold, the processing device 120 may determine that the data set execution result satisfies the execution condition, and the state of the medical device satisfies the preset condition. As another example, the processing device 120 may determine whether the state of the medical device satisfies the preset condition based on a difference between the measured dose distribution and the planned dose distribution. In response to determining that the difference between the measured dose distribution and the planned dose distribution is greater than a second difference threshold, the processing device 120 may determine that the data set execution result does not satisfy an execution condition, and the state of the medical device does not satisfy the preset condition. In response to determining that the difference between the measured dose distribution and the planned dose distribution is not greater than the second difference threshold, the processing device 120 may determine that the data set execution result satisfies the execution condition, and the state of the medical device satisfies the preset condition.

In some embodiments, the processing device 120 may determine whether the state of the medical device satisfies the preset condition based on a ratio of a number (or count) of data set execution results that meet the execution conditions to a total number (or count) of data set execution results. In response to determining that the ratio of the number (or count) of data set execution results that meet the execution conditions to the total number (or count) of data set execution results is greater than a first ratio threshold, the processing device 120 may determine that the state of the medical device satisfies the preset condition.

In some embodiments, the processing device 120 may determine a plurality of data set execution results (e.g., a plurality of differences between actual test results and predicted test results respectively) by directing the medical device to execute a same data set (e.g., the first data set, the second data set, the third data set) a plurality of times. The difference among the plurality of data set execution results may reflect a change of the state of the medical device. For example, during a delivery inspection of the medical device, a first execution result may be determined by directing the medical device to execute the third data set. After the medical device has been used for a period of time, a second execution result may be determined by directing the medical device to execute the third data set again. The difference between the first execution result and the second execution result may reflect a change of the state of the medical device between the two data set execution times.

In some embodiments, the processing device 120 may determine whether the state of the medical device satisfies the preset condition based on the plurality of data set execution results. For example, the processing device 120 may determine whether a difference between the first execution result and the second execution result is less than a third difference threshold. In response to determining that the difference between the first execution result and the second execution result is less than the third difference threshold, the processing device 120 may determine that the state of the medical device satisfies the preset condition.

In some embodiments, for the second data set or the third data set, the processing device 120 may determine whether the state of the medical device satisfies the preset condition based on a change degree of a ratio of a number (or count) of data set execution results that meet the execution conditions to a total number (or count) of data set execution results. For example, during the delivery inspection of the medical device, the processing device 120 may determine a plurality of first execution results by directing the medical device to execute the third data set a plurality of times. Then the processing device 120 may determine a first ratio of a number (or count) of first execution results that meet the execution conditions to a total number (or count) of first execution results. After the medical device has been used for a period of time, the processing device 120 may determine a plurality of second execution results by directing the medical device to execute the third data set a plurality of times. Then the processing device 120 may determine a second ratio of a number (or count) of second execution results that meet the execution conditions to a total number (or count) of second execution results. In response to determining that the difference between the first second ratio and the second ratio is less than a second ratio threshold, the processing device 120 may determine that the state of the medical device satisfies the preset condition.

In some embodiments, the first difference threshold, the second difference threshold, the third difference threshold, the first ratio threshold, and/or the second ratio threshold may be manually set by a user (e.g., a doctor, a technician) of the medical system 100, or automatically set by one or more components (e.g., the processing device 120) of the medical system 100 according to different situations.

In some embodiments, since the execution result of the first data set can reflect the accuracy to execute a common clinical radiotherapy plan using the medical device, a checking frequency of the state of the medical device based on the first data set may be relatively high (e.g., once a day, once a week). Since the execution result of the second data set and/or the execution result of the third data set can reflect the accuracy to execute an uncommon clinical radiotherapy plan using the medical device, a checking frequency of the state of the medical device based on the second data set and/or the third data set may be relatively low (e.g., once a month, once a year).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the first data set and/or the second data set may include a plurality of data subsets. The plurality of data subsets may correspond to different types of plans. For example, a radiotherapy plan may be classified according to a type of a target area (e.g., a tumor) of the target subject, a type of a radiotherapy technology (e.g., a two-dimensional conformal radiotherapy, a three-dimensional conformal radiotherapy, an intensity-modulated radiotherapy, an image-guided radiotherapy, a bio-guided radiotherapy, a dose-guided radiotherapy, an online adaptive radiotherapy) used in the radiotherapy plan, etc.

FIG. 7 is a flowchart illustrating an exemplary process for obtaining a state of a medical device according to some embodiments of the present disclosure. In some embodiments, process 700 may be executed by the medical system 100. For example, the process 700 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 700. The operations of the illustrated process presented below are intended to be illustrative.

In some embodiments, the process 700 may be accomplished with one or more additional operations not described and/or without one or more of the operations discussed. Additionally, the order of the operations of process 700 illustrated in FIG. 7 and described below is not intended to be limiting.

In 710, the processing device 120 (e.g., the first obtaining module 410) may obtain a data set, wherein the data set includes at least one sample parameter, each of which corresponds to at least one sample parameter value.

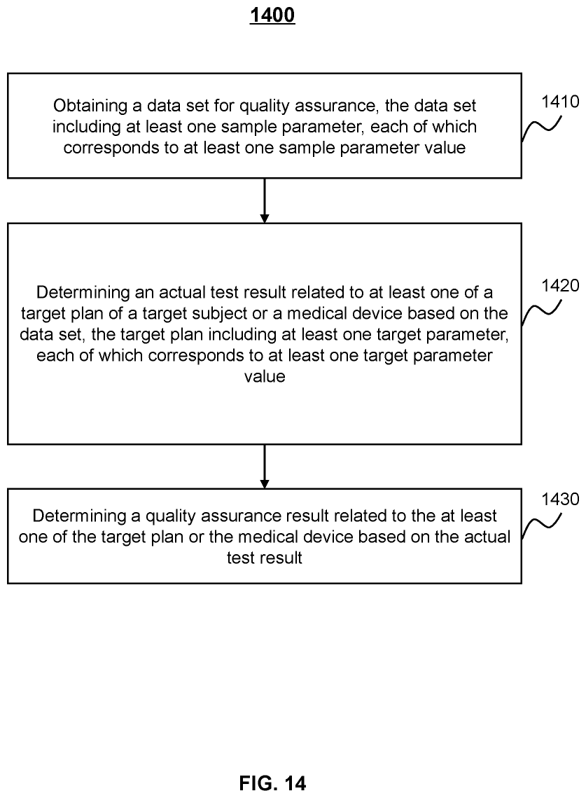

Operation 710 may be performed in a similar manner as operation 1410 as described in connection with FIG. 14, the descriptions of which are not repeated here.

In 720, the processing device 120 (e.g., the first obtaining module 410) may determine an actual test result related to at least one of a target plan of a target subject or a medical device by directing the medical device to execute the data set. The target plan may include at least one target parameter, each of which corresponds to at least one target parameter value. In some embodiments, at least part of the at least one target parameter may have the corresponding sample parameter in the data set. In some embodiments, for the target parameter value of each of the at least one target parameter, the target parameter value may be within or outside the parameter range of the sample parameter corresponding to the target parameter.

Operation 720 may be performed in a similar manner as operation 1420 as described in connection with FIG. 14, the descriptions of which are not repeated here.

In 730, the processing device 120 (e.g., the first obtaining module 410) may determine a data set execution result based on the actual test result.

In some embodiments, the processing device 120 may determine a predicted test result based on the data set using a test model. The processing device 120 may determine the data set execution result based on the predicted test result and the actual test result. Operation 730 may be performed in a similar manner as operation 1430 as described in connection with FIG. 14, the descriptions of which are not repeated here.

In 740, the processing device 120 (e.g., the first obtaining module 410) may obtain a state of the medical device based on the data set execution result.

In some embodiments, the data set execution result may reflect the state of the medical device. For example, the smaller the difference between the actual test result and the predicted test result is, the better the state of the medical device may be, and the higher the accuracy to execute a radiotherapy plan using the medical device may be. Operation 740 may be performed in a similar manner as operation 620 as described in connection with FIG. 6A, the descriptions of which are not repeated here.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 8 is a flowchart illustrating an exemplary process for determining a prediction result according to some embodiments of the present disclosure. In some embodiments, process 800 may be executed by the medical system 100. For example, the process 800 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 800. The operations of the illustrated process presented below are intended to be illustrative. In some embodiments, the process 800 may be accomplished with one or more additional operations not described and/or without one or more of the operations discussed. Additionally, the order of the operations of process 800 illustrated in FIG. 8 and described below is not intended to be limiting.

In 810, the processing device 120 (e.g., the first determination module 430) may determine at least one of feature information related to a complexity level of a target plan or a target fluence map based on the target plan of a target subject.

In some embodiments, the complexity level of the target plan may reflect a difficulty degree of a radiotherapy operation performed on the target subject based on value(s) of radiotherapy parameter(s) in the target plan. In some embodiments, the complexity level of the target plan may be related to the value(s) of the radiotherapy parameter(s) in the target plan. For example, the feature information related to the complexity level of the target plan may include a value of at least one radiotherapy parameter in the target plan, a change degree of the value of the at least one radiotherapy parameter in the target plan, a change rate of the value of the at least one radiotherapy parameter in the target plan, or the like, or any combination thereof.

In some embodiments, for some types of radiotherapy parameters, the larger the value of the radiotherapy parameter in the target plan is, the higher the complexity level of the target plan may be. For example, the larger the number (or count) of sub-fields in the target plan is, the higher the complexity level of the target plan may be. In some embodiments, for some types of radiotherapy parameters, the smaller the value of the radiotherapy parameter in the target plan is, the higher the complexity level of the target plan may be. For example, the smaller the area of the sub-field is, the higher the complexity level of the target plan may be. In some embodiments, the higher the change degree of the value of the radiotherapy parameter in the target plan is, the higher the complexity level of the target plan may be. For example, the higher the change degree of the dose rate during the radiotherapy process associated with the target plan is, the higher the complexity level of the target plan may be. In some embodiments, the higher the change rate of the value of the radiotherapy parameter in the target plan is, the higher the complexity level of the target plan may be. For example, the faster the gantry angle changes during the radiotherapy process associated with the target plan, the higher the complexity level of the target plan may be.

In some embodiments, the feature information related to the complexity level of the target plan may also include an irregularity of a radiation beam generated by the medical device, a product of a number (or count) of monitor units (MUs) corresponding to a radiation field and an area of the radiation field, a perimeter of the radiation field, a proportion of small sub-fields, a deviation degree of the radiation field (or sub-field) from a center point of the medical device, a deviation degree of the radiation field (or sub-field) from a beam center point, a change degree of positions of leaves of a collimator between adjacent sub-fields, a ratio of an area to a perimeter of the radiation field (or sub-field), a number (or count) of unconnected areas in the radiation field, or the like, or any combination thereof.

The proportion of small sub-fields may refer to, during a radiation field generation process, a proportion of a number (or count) of leaves of the collimator with sub-field areas less than a threshold (e.g., 1 cm, 2 cm) to a total number (or count) of leaves of the collimator involved in the radiation field generation process. For example, if ten pairs of leaves of the collimator are involved in the radiation field generation process, and a sub-field area of one pair of leaves of the collimator is less than the threshold (e.g., 2 cm), the proportion of small sub-fields may be determined as 1/10.

In some embodiments, the higher the irregularity of the radiation beam is, the greater the product of the number (or count) of monitor units (MUs) corresponding to the radiation field and the area of the radiation field is, the smaller the perimeter of the radiation field is, the higher the proportion of small sub-fields is, the higher the deviation degree of the radiation field from the center point of the medical device is, and/or the greater the change degree of positions of the leaves of the collimator between adjacent sub-fields is, the higher the complexity level of the target plan may be.

In some embodiments, the processing device 120 may determine the target fluence map based on the target plan of the target subject. In some embodiments, the target fluence map may be used to characterize a flux distribution of a radiation beam flow passing through the collimator. For example, the target fluence map may represent an expected radiation intensity distribution of the radiation beam to be delivered to a target area of the subject in the radiotherapy process. In some embodiments, the target fluence map may be determined based on the value(s) of the radiotherapy parameter(s) in the target plan using a dose model. The target fluence map may reflect information related to the radiotherapy parameter(s) (e.g., parameters related to the radiation field or the sub-field) and the parameter information of the dose model.

In some embodiments, for each radiation beam generated by the medical device, a radiotherapy operation may be performed on the target subject from a plurality of gantry angles based on the target plan of the subject. For each gantry angle of the plurality of gantry angles, the processing device 120 may determine a plurality of first sub-fluence maps corresponding to a plurality of sub-fields corresponding to the gantry angle based on a radiation intensity in a unit time corresponding to each sub-field of the plurality of sub-fields and a radiation time corresponding to each sub-field of the plurality of sub-fields. The processing device 120 may determine a second sub-fluence map corresponding to the gantry angle based on the plurality of first sub-fluence maps. For example, the processing device 120 may determine the second sub-fluence map by performing a weighted fusion operation on the plurality of first sub-fluence maps. Further, the processing device 120 may determine the target fluence map corresponding to the radiation beam based on a plurality of second sub-fluence maps corresponding to the plurality of gantry angles. For example, the processing device 120 may determine the target fluence map corresponding to the radiation beam by performing a weighted fusion operation on the plurality of second sub-fluence maps corresponding to the plurality of gantry angles.

In 820, the processing device 120 (e.g., the first determination module 430) may determine a prediction result based on a state of a medical device and the at least one of the feature information related to the complexity level of the target plan or the fluence map.

In some embodiments, the processing device 120 may input the state of the medical device, and the at least one of the feature information related to the complexity level of the target plan or the fluence map into a first model, and the first model may output the prediction result. More descriptions for determining the prediction result may be found elsewhere in the present disclosure (e.g., operation 530 in FIG. 5, and descriptions thereof).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 9 is a flowchart illustrating an exemplary process for performing a quality assurance test according to some embodiments of the present disclosure. In some embodiments, process 900 may be executed by the medical system 100. For example, the process 900 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 900. The operations of the illustrated process presented below are intended to be illustrative.

In some embodiments, the process 900 may be accomplished with one or more additional operations not described and/or without one or more of the operations discussed. Additionally, the order of the operations of process 900 illustrated in FIG. 9 and described below is not intended to be limiting.

In 910, the processing device 120 (e.g., the first determination module 430) may determine a prediction result based on a state of a medical device and a target plan of a target subject using a first model.

In some embodiments, the processing device 120 may input the state of the medical device, the target plan of the target subject (e.g., feature information related to a complexity level of the target plan, a target fluence map), parameter information of a dose model, a medical image of the target subject, or the like, or any combination thereof, into the first model, and the first model may output a predicted image (e.g., a predicted EPID image) of the target subject, a predicted gamma passing rate, a predicted dose distribution, or the like, or any combination thereof.

In some embodiments, the processing device 120 may determine a first feature vector based on the state of the medical device. For example, the processing device 120 may determine a systematic error and a random error of at least one component of the medical device as feature values in the first feature vector respectively. As another example, the processing device 120 may determine beam and dose information (e.g., a beam flatness, a beam symmetry, a beam linearity, a beam output correction, a beam output consistency), positioning accuracy information of the at least one component of the medical device, operation error information of the at least one component of the medical device, and a data set execution result as feature values in the first feature vector respectively. The processing device 120 may determine a second feature vector based on the target plan of the target subject. For example, the processing device 120 may determine the feature information related to the complexity level of the target plan and the target fluence map as feature values in the second feature vector respectively.

Further, the processing device 120 may input the first feature vector and the second feature vector into the first model, and the first model may output the predicted image (e.g., the predicted EPID image) of the target subject, the predicted gamma passing rate, and/or the predicted dose distribution. For example, the first model may output the predicted image (e.g., the predicted EPID image). The processing device 120 may determine the predicted gamma passing rate based on the predicted image (e.g., the predicted EPID image) and a calculate image (e.g., a calculate EPID image) according to a gamma analysis method. As another example, the first model may output the predicted gamma passing rate directly.

In some embodiments, the processing device 120 may input a first target fluence map into a first feature extraction model (e.g., a 2D feature extraction model). The first target fluence map may be determined based on a medical device without systematic error and/or random error. The first feature extraction model may determine a 2D feature vector corresponding to the first target fluence map. The processing device 120 may input the systematic error and/or the random error of the at least one component of the medical device into a second feature extraction model (e.g., a 1D feature extraction model). The second feature extraction model may determine a 1D feature vector corresponding to the systematic error and/or the random error. The processing device 120 may input the 1D feature vector corresponding to the systematic error and/or the random error into a dimension conversion model. The dimension conversion model may convert the 1D feature vector corresponding to the systematic error and/or the random error into a 2D feature vector corresponding to the systematic error and/or the random error. Further, the processing device 120 may input the 2D feature vector corresponding to the first target fluence map and the 2D feature vector corresponding to the systematic error and/or the random error into the first model. The first model may output the predicted image (e.g., the predicted EPID image).

As used herein, a first feature extraction model (or the second feature extraction model) refers to an algorithm or process configured to determine a feature vector based on input information. A dimension transformation model refers to an algorithm or process configured to transform data dimensions (e.g., a transformation between 1D feature vector, 2D feature vector, and 3D feature vector).

In some embodiments, the processing device 120 may input a second target fluence map into the first feature extraction model (e.g., the 2D feature extraction model). The second target fluence map may be determined based on a medical device with systematic error and/or random error. The first feature extraction model may determine a 2D feature vector corresponding to the second target fluence map. Further, the processing device 120 may input the 2D feature vector corresponding to the second target fluence map into the first model. The first model may output the predicted image (e.g., the predicted EPID image).

In some embodiments, the processing device 120 may input the first target fluence map into the first feature extraction model (e.g., the 2D feature extraction model). The first target fluence map may be determined based on the medical device without systematic error and/or random error. The first feature extraction model may determine the 2D feature vector corresponding to the first target fluence map. The processing device 120 may input the 2D feature vector corresponding to the first target fluence map into the dimension conversion model. The dimension conversion model may convert the 2D feature vector corresponding to the first target fluence map into a 1D feature vector corresponding to the first target fluence map. The processing device 120 may input the systematic error and/or the random error of the at least one component of the medical device, the feature information related to the complexity level of the target plan, and/or the parameter information of the dose model into the second feature extraction model (e.g., a 1D feature extraction model) respectively. The second feature extraction model may determine a 1D feature vector corresponding to the systematic error and/or the random error, a 1D feature vector corresponding to the feature information related to the complexity level of the target plan, and/or a 1D feature vector corresponding to the parameter information of the dose model respectively. Further, the processing device 120 may input the 1D feature vector corresponding to the first target fluence map, the 1D feature vector corresponding to the systematic error and/or the random error, the 1D feature vector corresponding to the feature information related to the complexity level of the target plan, and/or the 1D feature vector corresponding to the parameter information of the dose model into the first model. The first model may output the predicted image (e.g., the predicted EPID image).

In the present disclosure, the input (e.g., the state of the medical device, the target plan of the target subject, the parameter information of the dose model) of the first model in operation 610 may also be referred to as an initial input (e.g., an initial state of the medical device, an initial target plan, initial parameter information of the dose model) of the first model. The prediction result (e.g., the predicted image, the predicted gamma passing rate, the predicted dose distribution) determined in operation 610 may also be referred to as an initial prediction result (e.g., an initial predicted image, an initial predicted gamma passing rate, an initial predicted dose distribution).

In 920, the processing device 120 (e.g., the second determination module 440) may determine whether a quality assurance test passes based on the prediction result.

Operation 920 may be performed in a similar manner as operation 540 as described in connection with FIG. 5, the descriptions of which are not repeated here.

In response to determining that the quality assurance test passes, process 900 may proceed to operation 980.

In response to determining that the quality assurance test does not pass, process 900 may proceed to operation 930.

In 930, the processing device 120 (e.g., the second determination module 440) may determine a reason that the quality assurance test does not pass based on the state of the medical device, the target plan of the target subject, and the prediction result using a second model.

In some embodiments, the processing device 120 may input the state of the medical device, the target plan of the target subject, the parameter information of the dose model, and the prediction result (e.g., the predicted image) into the second model, and the second model may output the reason that the quality assurance test does not pass.

The reason that the quality assurance test does not pass may include a reason related to the medical device, a reason related to the target plan, a reason related to the dose model, or the like, or any combination thereof. The reason related to the medical device may include a relatively large operation error of at least one component of the medical device (e.g., a relatively large systematic error and/or a relatively large random error of a multi-leaf collimator), or the like, or any combination thereof. The reason related to the target plan may include a relatively high complexity level of the target plan (e.g., a relatively small area of a radiation field, a relatively high degree deviation of a radiation field from an isocenter of the medical device), or the like, or any combination thereof. The reason related to the dose model may include an inappropriate type of the dose model, an inappropriate parameter of the dose model, or the like, or any combination thereof.

In some embodiments, the second model may output a plurality of reasons that the quality assurance test does not pass and/or a ranking of the plurality of reasons that the quality assurance test does not pass. For example, the plurality of reasons that the quality assurance test does not pass may be ranked according to their probabilities of causing the quality assurance test does not pass in a descending order.

In some embodiments, the processing device 120 may determine a first feature vector based on the state of the medical device. The processing device 120 may determine a second feature vector based on the target plan. The processing device 120 may determine a third feature vector based on the parameter information of the dose model. The processing device 120 may determine a fourth feature vector based on the predicted image. Then the processing device 120 may determine the reason that the quality assurance test does not pass based on the first feature vector, the second feature vector, the third feature vector, and the fourth feature vector using the second model. For example, the processing device 120 may input the first feature vector, the second feature vector, the third feature vector, and the fourth feature vector into the second model. The second model may output the reason that the quality assurance test does not pass. As another example, the processing device 120 may input the first feature vector, the second feature vector, and the fourth feature vector into the second model, and the second model may output the reason that the quality assurance test does not pass.

In some embodiments, the processing device 120 may input the first target fluence map into the first feature extraction model (e.g., the 2D feature extraction model). The first target fluence map may be determined based on the medical device without systematic error and/or random error. The first feature extraction model may determine the 2D feature vector corresponding to the first target fluence map. The processing device 120 may input the 2D feature vector corresponding to the first target fluence map into the dimension conversion model. The dimension conversion model may convert the 2D feature vector corresponding to the first target fluence map into a 1D feature vector corresponding to the first target fluence map. The processing device 120 may input the predicted image into the first feature extraction model (e.g., the 2D feature extraction model). The first feature extraction model may determine the 2D feature vector corresponding to the predicted image. The processing device 120 may input the 2D feature vector corresponding to the predicted image into the dimension conversion model. The dimension conversion model may convert the 2D feature vector corresponding to the predicted image into a 1D feature vector corresponding to the predicted image. The processing device 120 may input the systematic error and/or the random error of the at least one component of the medical device, the feature information related to the complexity level of the target plan, and the parameter information of the dose model into the second feature extraction model (e.g., a 1D feature extraction model) respectively. The second feature extraction model may determine a 1D feature vector corresponding to the systematic error and/or the random error, a 1D feature vector corresponding to the feature information related to the complexity level of the target plan, and a 1D feature vector corresponding to the parameter information of the dose model respectively.

Further, the processing device 120 may input the 1D feature vector corresponding to the first target fluence map, the 1D feature vector corresponding to the predicted image, the 1D feature vector corresponding to the systematic error and/or the random error, the 1D feature vector corresponding to the feature information related to the complexity level of the target plan, and the 1D feature vector corresponding to the parameter information of the dose model into the second model. The second model may output the reason that the quality assurance test does not pass.

In 940, the processing device 120 (e.g., the second determination module 440) may adjust, based on the reason that the quality assurance test does not pass, a value of a parameter associated with at least one of the medical device, the target plan, or a dose model.

In some embodiments, if the reason that the quality assurance test does not pass is the reason related to the medical device, the processing device 120 or a user (e.g., a doctor) of the medical system 100 may adjust value(s) of parameter(s) associated with the medical device. For example, at least one component of the medical device may be calibrated to improve the positioning accuracy of the at least one component and/or reduce the systematic error and/or the random error of the at least one component.

In some embodiments, if the reason that the quality assurance test does not pass is the reason related to the target plan, the processing device 120 or the user of the medical system 100 may adjust value(s) of parameter(s) associated with the target plan. For example, the value(s) of the parameter(s) in the target plan may be increased or decreased to reduce the complexity level of the target plan.

In some embodiments, if the reason that the quality assurance test does not pass is the reason related to the dose model, the processing device 120 or the user of the medical system 100 may adjust value(s) of parameter(s) associated with the dose model. For example, the value(s) of the parameter(s) in the dose model may be adjusted, or another type of dose model may be used.

In some embodiments, when the second model outputs a plurality of reasons that the quality assurance test does not pass, the processing device 120 (or the user) may adjust values of parameters associated with one or more reasons that the quality assurance test does not pass in the plurality of reasons that the quality assurance test does not pass.

In 950, the processing device 120 (e.g., the first determination module 430) may determine an updated prediction result based on an adjusted value of the parameter using the first model.

In some embodiments, the processing device 120 may input the adjusted value of the parameter and other initial input(s) (e.g., the initial input of the first model as described in operation 910) not adjusted in operation 940 into the first model, and the first model may output an updated predicted image (e.g., an updated predicted EPID image) of the target subject, an updated gamma passing rate, and/or an updated dose distribution.

For example, if the reason that the quality assurance test does not pass is the reason related to the medical device, the processing device 120 may input an adjusted state of the medical device (determined based on the adjusted value of the parameter associated with the medical device) and the initial target plan into the first model, and the first model may output the updated predicted image, the updated gamma passing rate, and/or the updated dose distribution. As another example, if the reason that the quality assurance test does not pass is the reason related to the target plan, the processing device 120 may input an adjusted target plan (determined based on the adjusted value of the parameter associated with the target plan) and the initial state of the medical device into the first model, and the first model may output the updated predicted image, the updated gamma passing rate, and/or the updated dose distribution.

In some embodiments, the processing device 120 may determine a target adjustment mode based on the updated prediction result. In some embodiments, if the updated prediction result is better than the initial prediction result, the processing device 120 may determine a current adjustment mode (i.e., a parameter adjustment mode as described in operation 940) as the target adjustment mode. For example, if the reason that the quality assurance test does not pass is the reason related to the target plan, the processing equipment 120 may decrease an area of a radiation field in the target plan, and input the adjusted target plan and other initial input(s) not adjusted into the first model. The first model may output an updated gamma passing rate. In response to determining that the updated gamma passing rate is greater than the initial gamma passing rate, the processing device 120 may determine that the target adjustment mode is decreasing the area of the radiation field in the target plan. In some embodiments, in response to determining that the updated gamma passing rate is not greater than the initial gamma passing rate, the processing device 120 may determine that the target adjustment mode is increasing the area of the radiation field in the target plan. In some embodiments, in response to determining that the updated gamma passing rate is not greater than the initial gamma passing rate, the processing device 120 may adjust a value of other parameter in the target plan, or a value of a parameter associated with the medical device or the dose model.

In 960, the processing device 120 (e.g., the second determination module 440) may determine whether the quality assurance test passes based on the updated prediction result.

Operation 960 may be performed in a similar manner as operation 920 as described in connection with FIG. 9, the descriptions of which are not repeated here.

In response to determining that the quality assurance test passes, process 900 may proceed to operation 970.

In response to determining that the quality assurance test does not pass, process 900 may proceed to operation 930 to further determine a reason that the quality assurance test does not pass based on the updated prediction result. In some embodiments, the iteration of operation 930 to operation 960 may be repeated until the quality assurance test passes.

In 970, the processing device 120 (e.g., the second determination module 440) may control the medical device to treat and/or scan the target subject according to an updated target plan.

The updated target plan may be determined based on the adjusted value of the parameter. For example, the processing device 120 may control at least one component of the medical device to perform a radiotherapy operation on the target subject based on adjusted value(s) of radiotherapy parameter(s) in the updated target plan of the target subject.

In 980, the processing device 120 (e.g., the second determination module 440) may control the medical device to treat and/or scan the target subject according to the target plan.

For example, the processing device 120 may control the at least one component of the medical device to perform the radiotherapy operation on the target subject based on value(s) of radiotherapy parameter(s) in the target plan of the target subject.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 120 may generate a reminder. The reminder may be in the form of text, voice, a picture, a video, a haptic alert, or the like, or any combination thereof. In some embodiments, the reminder may include information regarding a quality assurance test result (whether the quality assurance test passes). In some embodiments, in response to determining that the quality assurance test does not pass, the reminder may include the reason that the quality assurance test does not pass and/or the target adjustment mode.

In some embodiments, an output layer of the first model may be connected with an input layer of the second model. The processing device 120 may input the state of the medical device, the target plan of the target subject, the medical image of the target subject, and the parameter information of the dose model into an input layer of the first model. The prediction result determined by the first model and the input of the first model may be directly input into the input layer of the second model. The second model may output the prediction result and the reason that the quality assurance test does not pass. For example, the first model may determine whether the quality assurance test passes based on the prediction result. In response to determining that the quality assurance test passes, the first model may output the prediction result. In response to determining that the quality assurance test does not pass, the first model may input the prediction result and the input of the first model into the input layer of the second model. The second model may output the prediction result and the reason that the quality assurance test does not pass.

In some embodiments, the first model and second model may be two separated models, or combined into a single model.

FIG. 10 is a flowchart illustrating an exemplary process for determining a first model and a second model according to some embodiments of the present disclosure. In some embodiments, process 1000 may be executed by the medical system 100. For example, the process 1000 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 4) may execute the set of instructions and may accordingly be directed to perform the process 1000. The operations of the illustrated process presented below are intended to be illustrative. In some embodiments, the process 900 may be accomplished with one or more additional operations not described and/or without one or more of the operations discussed. Additionally, the order of the operations of process 1000 illustrated in FIG. 10 and described below is not intended to be limiting.

In 1010, the processing device 120 (e.g., the first determination module 430) may obtain a plurality of groups of training samples.

The plurality of groups of training samples may be used to train a first model and a second model. In some embodiments, each group of training samples may include a sample state of a medical device, a sample plan of a sample subject (e.g., sample feature information related to a complexity level of the sample plan and/or a sample fluence map determined based on the sample plan), sample parameter information of a dose model, a reference image (e.g., a reference EPID image), a reference reason that the quality assurance test does not pass, or the like, or a combination thereof. As used herein, a sample subject refers to a subject whose data is used for training the first model and the second model. The reference image and the reference reason that the quality assurance test does not pass may be used as gold standards for model training. The sample state of a medical device may include beam and dose information, positioning accuracy information of at least one component (e.g., a gantry, a radiation source, a collimator) of the medical device, operation error information of the at least one component of the medical device, data set execution result, or the like, or any combination thereof, as described in connection with operation 510. The sample parameter information of the dose model may include a type of the dose model, a parameter of the dose model, or the like, or any combination thereof, as described in connection with operation 610.

In some embodiments, the group of training samples may include historical data related to a historical scan process of the sample subject. For example, the group of training samples may include a historical state of the medical device, a historical plan of the sample subject, historical parameter information of the dose model, a historical image (e.g., a historical EPID image) of the sample subject, a historical reason that the quality assurance test does not pass, or the like, or any combination thereof.

In some embodiments, the plurality of groups of training samples or a portion thereof may include simulated data. For example, the processing device 120 or a user (e.g., a doctor) of the medical system 100 may determine the simulated data by adjusting the historical data related to the historical scan process of the sample subject. For example, the sample plan may be determined by adjusting value(s) of parameter(s) in the historical plan, to obtain the sample plan with an expected complexity level (e.g., a relatively high complexity level). By using the simulated data as the training samples, the diversity of the training samples can be improved, and the accuracy and effectiveness of the model trained based on the training samples can be improved.

In some embodiments, the first model and the second model may be determined by performing a plurality of iterations to iteratively update one or more parameter values of a first preliminary model and a second preliminary model. The parameters of the first preliminary model and the second preliminary model may include a size of a core of a layer, a total number (or count) of layers, a number (or count) of nodes in each layer, a connected weight between two connected nodes, a deviation vector related to nodes, or the like, or any combination thereof. In each iteration, one or more groups of training samples selected from the plurality of groups of training samples may be used to jointly train the first preliminary model and the second preliminary model to generate the first model and the second model. Operations 1020-1070 may be an example of an iteration.

In 1020, the processing device 120 (e.g., the first determination module 430) may obtain a candidate image by inputting a group of training samples into a first preliminary model.

As used herein, a first preliminary model refers to a machine learning model to be trained to generate the first model. In some embodiments, the processing device 120 may initialize one or more parameter values of one or more parameters in the first preliminary model. In some embodiments, the initialized values of the parameters may be default values determined by the medical system 100 or preset by a user of the medical system 100. In some embodiments, the processing device 120 may obtain the first preliminary model from a storage device (e.g., the storage device 130) of the medical system 100 and/or an external storage device via the network 150.

In some embodiments, the processing device 120 may input the sample state of the medical device, the sample plan of the sample subject, and the sample parameter information of the dose model in the group of training samples into the first preliminary model. The first preliminary model may determine a first predicted output (e.g., the candidate image) based on the sample state of the medical device, the sample plan of the sample subject, and the sample parameter information of the dose model.

In 1030, the processing device 120 (e.g., the first determination module 430) may obtain a candidate reason that the quality assurance test does not pass by inputting the group of training samples and the candidate image into a second preliminary model.

As used herein, a second preliminary model refers to a machine learning model to be trained to generate the second model. In some embodiments, the processing device 120 may determine the second preliminary model in a manner similar as that of the first preliminary model. In some embodiments, the sample state of the medical device, the sample plan of the sample subject, the sample parameter information of the dose model in the group of training samples and the candidate image may be input into the second preliminary model. The second preliminary model may determine a second predicted output (e.g., the candidate reason that the quality assurance test does not pass) based on the sample state of the medical device, the sample plan of the sample subject, the sample parameter information of the dose model, and the candidate image.

In 1040, the processing device 120 (e.g., the first determination module 430) may determine a first loss function value based on the candidate image and a reference image.

As used herein, a first loss function may be configured to assess a difference between the first predicted output (e.g., the candidate image) of the first preliminary model and a desired output (e.g., the reference image) of the first preliminary model.

In 1050, the processing device 120 (e.g., the first determination module 430) may determine a second loss function value based on the candidate reason that the quality assurance test does not pass and a reference reason that the quality assurance test does not pass.

As used herein, a second loss function may be configured to assess a difference between the second predicted output (e.g., the candidate reason that the quality assurance test does not pass) of the second preliminary model and a desired output (e.g., the reference reason that the quality assurance test does not pass) of the second preliminary model.

In 1060, the processing device 120 (e.g., the first determination module 430) may determine a target loss function value based on the first loss function value and the second loss function value.

In some embodiments, the processing device 120 may obtain a first weight corresponding to the first loss function and a second weight corresponding to the second loss function. The first weight (or the second weight) may indicate an importance of the first loss function (or the second loss function) in a joint training of the first preliminary model and the second preliminary model. The processing device 120 may determine the target loss function value based on the first loss function value, the first weight corresponding to the first loss function, the second loss function value, and the second weight corresponding to the second loss function. For example, assuming that the first loss function value is A1, the second loss function value is A2, the first weight corresponding to the first loss function is W1, and the second weight corresponding to the second loss function is W2, the processing device 120 may determine the target loss function value is A=A1×W1+A2×W2.

In some embodiments, the first weight and the second weight may be manually set by a user (e.g., a doctor, a technician) of the medical system 100, or automatically set by one or more components (e.g., the processing device 120) of the medical system 100 according to different situations.

In 1070, the processing device 120 (e.g., the first determination module 430) may update at least one of the first preliminary model or the second preliminary model based on the target loss function value to generate a first model and a second model.

In some embodiments, parameter values of the first preliminary model and/or the second preliminary model may be adjusted and/or updated in order to decrease the target loss function value, and an updated first preliminary model and/or an updated second preliminary model may be generated. Accordingly, in a next iteration, one or more groups of training samples of the plurality of groups of training samples may be input into the updated first preliminary model and/or the updated second preliminary model to train the updated first preliminary model and/or the updated second preliminary model as described above. The group(s) of training samples used in different iterations may be the same or different.

In some embodiments, the plurality of iterations may be performed to update the parameter values of the first preliminary model and/or the second preliminary model until a termination condition is satisfied. The termination condition may provide an indication of whether the first preliminary model and/or the second preliminary model are sufficiently trained. The termination condition may relate to the target loss function or an iteration count of the iterative process or training process. For example, the termination condition may be satisfied if the target loss function value is minimal or smaller than a threshold (e.g., a constant). As another example, the termination condition may be satisfied if the value of the target loss function value converges. The convergence may be deemed to have occurred if the variation of the target loss function values in two or more consecutive iterations is smaller than a threshold (e.g., a constant). As still another example, the termination condition may be satisfied when a specified number (or count) of iterations are performed in the training process.

In response to determining that the termination condition is satisfied, the processing device 120 may designate the first preliminary model (or the updated first preliminary model) and the second preliminary model (or the updated second preliminary model) in the current iteration as the first model and the second model respectively.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, in operation 1040, in response to determining that the first loss function value is greater than a first threshold, parameter values of the first preliminary model may be adjusted and/or updated in order to decrease the first loss function value. In some embodiments, in operation 1050, in response to determining that the second loss function value is greater than a second threshold, parameter values of the second preliminary model may be adjusted and/or updated in order to decrease the second loss function value. The first threshold and the second threshold may be manually set by a user (e.g., a doctor, a technician) of the medical system 100, or automatically set by one or more components (e.g., the processing device 120) of the medical system 100 according to different situations.

In some embodiments, operation 1040 may be omitted. That is, the target loss function value may be the second loss function value. The processing device 120 may jointly train the first preliminary model and the second preliminary model based on the second loss function value.

In some embodiments, the processing device 120 may train the first preliminary model and the second preliminary model separately to generate the first model and the second model respectively. Take a training process of the first preliminary model as an example, the processing device 120 may obtain a plurality of groups of first training samples. Each group of first training samples may include a sample state of a medical device, a sample plan of a sample subject, and a reference prediction result. The reference prediction result may include a reference image and/or a reference gamma passing rate. The first model may be determined by performing a plurality of iterations to iteratively update one or more parameter values of a first preliminary model using the plurality of groups of first training samples. In a first iteration, the processing device 120 may obtain the first preliminary model. In subsequent iterations, the processing device 120 may obtain an updated first preliminary model generated in a previous iteration. The processing device 120 may input the sample state of the medical device and the sample plan of the sample subject in a group of first training samples into an input layer of the first preliminary model (or the updated first preliminary model), and input the reference prediction result in the group of first training samples into an output layer of the first preliminary model (or the updated first preliminary model). The first preliminary model (or the updated first preliminary model) may output a candidate prediction result including a candidate image and/or a candidate gamma passing rate. A loss function value may be determined based on a difference between the reference prediction result and the candidate prediction result. In some embodiments, the plurality of iterations may be performed to update the one or more parameter values of the first preliminary model (or the updated first preliminary model) until a termination condition is satisfied. The termination condition may relate to the loss function value or an iteration count of the iterative process or training process. In response to determining that the termination condition is satisfied, the processing device 120 may designate the first preliminary model (or the updated first preliminary model) in the current iteration as the first model. In some embodiments, in response to determining that the termination condition does not satisfied, one or more parameter values of the first preliminary model (or the updated first preliminary model) may be adjusted and/or updated until the termination condition is satisfied. Similarly, the processing device 120 may obtain a plurality of groups of second training samples. Each group of first training samples may include the sample state of the medical device, the sample plan of the sample subject, a sample prediction result (e.g., a sample predicted image), and a reference reason that the quality assurance test does not pass. The second model may be determined by performing a plurality of iterations to iteratively update one or more parameter values of a second preliminary model using the plurality of groups of second training samples.

It should be noted that the above description is merely provided for the purposes of illustration, and not intended to limit the scope of the present disclosure. The plurality of groups of training samples in operation 1010, the plurality of groups of first training samples, and/or the plurality of groups of second training samples may include other information or without at least part of the information discussed. For example, the group of first training samples and/or the group of second training samples may include the sample parameter information of the dose model, the sample feature information related to the complexity level of the sample plan, the sample fluence map, or the like, or any combination thereof. In some embodiments, in each iteration, one or more groups of first training samples (or one or more groups of second training samples) may be used to train the first preliminary model (or the second preliminary model) to generate the first model (or the second model).

In some embodiments, the processing device 120 may train the first preliminary model and the second preliminary model separately to generate a first candidate model and a second candidate model respectively. Further, the processing device 120 may jointly train the first candidate model and the second candidate model to generate the first model and the second model respectively. In some embodiments, the processing device 120 may jointly train the first preliminary model and the second preliminary model to generate a third candidate model and a fourth candidate model respectively. Further, the processing device 120 may train the third candidate model and the fourth candidate model separately to generate the first model and the second model respectively.

In some embodiments, the generation, training, and/or updating of the first model and/or the second model may be performed on a processing device, while the application of the first model and/or the second model may be performed on a different processing device. In some embodiments, the generation and/or updating of the first model and/or the second model may be performed on a processing device of a system different from the medical system 100 or a server different from a server including the processing device 120 on which the application of the first model and/or the second model is performed. For instance, the generation and/or updating of the first model and/or the second model may be performed on a first system of a vendor who provides and/or maintains such a first model and/or a second model and/or has access to training samples used to generate the first model and/or the second model while predicted result determination based on the provided first model and/or the second model may be performed on a second system of a client of the vendor. In some embodiments, the generation and/or updating of the first model and/or the second model may be performed on a first processing device of the medical system 100, while the application of the first model and/or the second model may be performed on a second processing device of the medical system 100. In some embodiments, the generation and/or updating of the first model and/or the second model may be performed online in response to a request for predicted result determination. In some embodiments, the generation and/or updating of the first model and/or the second model may be performed offline.

In some embodiments, the first model and/or the second model may be generated, trained, and/or updated (or maintained) by, e.g., the manufacturer of the medical device 110 or a vendor. For instance, the manufacturer or the vendor may load the first model and/or the second model into the medical system 100 or a portion thereof (e.g., the processing device 120) before or during the installation of the medical device 110 and/or the processing device 120, and maintain or update the first model and/or the second model from time to time (periodically or not). The maintenance or update may be achieved by installing a program stored on a storage device (e.g., a compact disc, a USB drive, etc.) or retrieved from an external source (e.g., a server maintained by the manufacturer or vendor) via the network 150. The program may include a new model (e.g., a new first model and/or a new second model) or a portion thereof that substitutes or supplements a corresponding portion of the first model and/or the second model.

According to some embodiments of the present disclosure, the state of medical device may change with the prolongation of the service time of medical device, and different states of the medical device may have a great impact on the quality assurance test result. Accordingly, by fully considering the current state of the medical device in the quality assurance test, the accuracy of the quality assurance test result can be improved. The data set execution results obtained by directing the medical device to execute different types of data sets (e.g., the first data set, the second data set, the third data set) can reflect the accuracy of medical device to execute different types of plans (e.g., radiotherapy plans), and the state of the medical device may be evaluated based on the data set execution results, which can simplify the quality assurance test and improve the efficiency of the quality assurance test. In addition, in the dose determination, in order to improve the efficiency of dose determination, the medical device may be modeled using the dose model. Different parameter information of the dose model may have a great impact on the quality assurance test result. Accordingly, by fully considering the parameter information of the dose model in the quality assurance test, the accuracy of the quality assurance test result can be improved. Moreover, the prediction result may be determined based on the state of the medical device, the target plan of the target subject, and the parameter information of the dose model using the first model, which can save time of the quality assurance test, improve the efficiency of the quality assurance test, and reduce the labor intensity of a user. The reason that the quality assurance test does not pass may be determined based on the state of the medical device, the target plan of the target subject, and the prediction result using the second model, which can save time of a user, and improve the efficiency of the radiotherapy process. Furthermore, the first model and/or the second model may be updated based on updated training samples. For example, the first model and/or the second model may be updated with the development of radiotherapy technology and the change of application scenarios, so as to ensure the rationality and accuracy of the quality assurance test based on the first model and/or the second model.

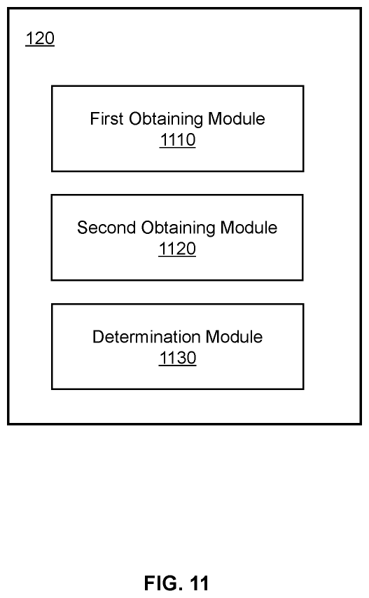

FIG. 11 is a schematic diagram illustrating an exemplary processing device according to some embodiments of the present disclosure. In some embodiments, the processing device 120 may include a first obtaining module 1110, a second obtaining module 1120, and a determination module 1130.

In some embodiments, the first obtaining module 1110 may be configured to obtain a state of a medical device. The second obtaining module 1120 may be configured to obtain a target plan of a target subject. The determination module 1130 may be configured to determine a prediction result based on the state of the medical device and the target plan of the target subject using a quality assurance model (e.g., a first model). The quality assurance model is a machine learning model.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the first obtaining module 1110 and the second obtaining module 1120 may be combined into a single module. In some embodiments, one or more modules may be added or omitted in the processing device 120. For example, the processing device 120 may further include a storage module (not shown in FIG. 11) configured to store data and/or information (e.g., a state of a medical device, a target plan of a target subject, a prediction result) associated with the medical system 100. As another example, the processing device 120 may further include a training module (not shown in FIG. 11) configured to train a model (e.g., the first model, the second model).

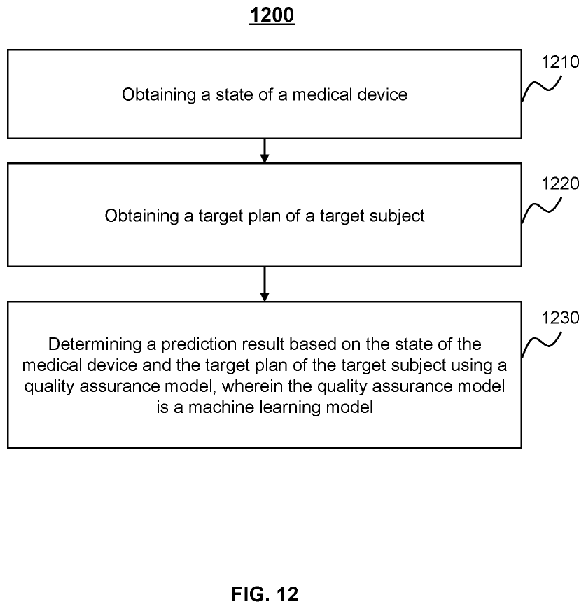

FIG. 12 is a flowchart illustrating an exemplary process for determining a prediction result according to some embodiments of the present disclosure. In some embodiments, process 1200 may be executed by the medical system 100. For example, the process 1200 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 11) may execute the set of instructions and may accordingly be directed to perform the process 1200. The operations of the illustrated process presented below are intended to be illustrative. In some embodiments, the process 1200 may be accomplished with one or more additional operations not described and/or without one or more of the operations discussed. Additionally, the order of the operations of process 1200 illustrated in FIG. 12 and described below is not intended to be limiting.

In 1210, the processing device 120 (e.g., the first obtaining module 1110) may obtain a state of a medical device.

Operation 1210 may be performed in a similar manner as operation 510 as described in connection with FIG. 5, the descriptions of which are not repeated here.

In 1220, the processing device 120 (e.g., the second obtaining module 1120) may obtain a target plan of a target subject.

Operation 1220 may be performed in a similar manner as operation 520 as described in connection with FIG. 5, the descriptions of which are not repeated here.

In 1230, the processing device 120 (e.g., the determination module 1130) may determine a prediction result based on the state of the medical device and the target plan of the target subject using a quality assurance model (e.g., a first model). The quality assurance model is a machine learning model.

Operation 1230 may be performed in a similar manner as operation 530 as described in connection with FIG. 5, the descriptions of which are not repeated here.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

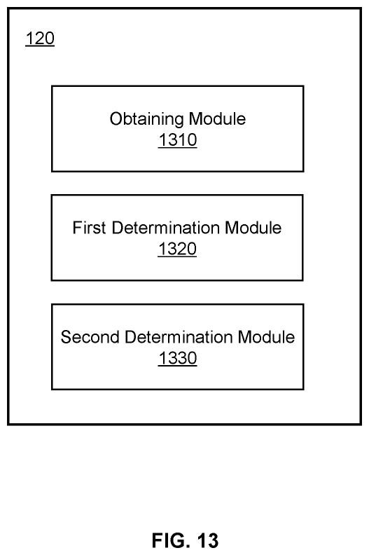

FIG. 13 is a schematic diagram illustrating an exemplary processing device according to some embodiments of the present disclosure. In some embodiments, the processing device 120 may include an obtaining module 1310, a first determination module 1320, and a second determination module 1330.

In some embodiments, the obtaining module 1310 may be configured to obtain a data set for quality assurance. The first determination module 1320 may be configured to determine an actual test result related to at least one of a target plan of a target subject or a medical device based on the data set. The second determination module 1330 may be configured to determine a quality assurance result related to the at least one of the target plan or the medical device based on the actual test result.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the first determination module 1320 and the second determination module 1330 may be combined into a single module. In some embodiments, one or more modules may be added or omitted in the processing device 120. For example, the processing device 120 may further include a storage module (not shown in FIG. 13) configured to store data and/or information (e.g., a data set, an actual test result, a quality assurance result) associated with the medical system 100.

FIG. 14 is a flowchart illustrating an exemplary process for determining a quality assurance result according to some embodiments of the present disclosure. In some embodiments, process 1400 may be executed by the medical system 100. For example, the process 1400 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 13) may execute the set of instructions and may accordingly be directed to perform the process 1400. The operations of the illustrated process presented below are intended to be illustrative. In some embodiments, the process 1400 may be accomplished with one or more additional operations not described and/or without one or more of the operations discussed. Additionally, the order of the operations of process 1400 illustrated in FIG. 14 and described below is not intended to be limiting.

In 1410, the processing device 120 (e.g., the obtaining module 1310) may obtain a data set for quality assurance.

In some embodiments, the data set may include at least one sample parameter, each of which corresponds to at least one sample parameter value. In some embodiments, for each sample parameter, the at least one sample parameter value of the sample parameter may define a parameter range. For example, the parameter range of a sample parameter may be smaller or equal to the maximum of the sample parameter values of the sample parameter. Merely by way of example, in the data set, if a sample parameter of a number (or count) of sub-fields corresponds to sample parameter values of 5, 6, 7, 8, 9, and 10, the parameter range of the number (or count) of sub-fields defined by the sample parameter values of the number (or count) of sub-fields is between 0 and 10. As another example, in the data set, if a sample parameter of a number (or count) of sub-fields corresponds to only one sample parameter value of 10, the parameter range of the number (or count) of sub-fields defined by the sample parameter value of the number (or count) of sub-fields may be between 0 and 10.

In some embodiments, the data set may be determined based on a plurality of sample plans (also can be referred to as “second sample plan” and the “sample plan” described in FIG. 10 also can be referred to as “first sample plan”), parameter information (e.g., limit performance information) of at least one component of the medical device, parameter information of a test model (e.g., a calculation limit of the test model), or the like, or any combination thereof.

In some embodiments, the data set may be determined based on the plurality of sample plans. Each sample plan may include a plurality of sample parameters and one or more sample parameter values corresponding to each of the plurality of sample parameters. The sample parameter may include a radiotherapy parameter associated with a radiotherapy process. For example, the sample parameter in the data set may include at least one radiotherapy parameter in the plurality of sample plans. The radiotherapy parameter may include a dose rate (e.g., MUs/min) of a radiation source (e.g., a treatment radiation source), a change rate of the dose rate, a radiation duration, a gantry angle (corresponding to a specific time period or time point), a gantry movement (e.g., rotation) speed (corresponding to a specific time period or time point), a gantry movement acceleration, a collimator angle (corresponding to a specific time period or time point), a collimator rotation speed (corresponding to a specific time period or time point), a parameter (e.g., a position, a movement speed, a movement acceleration, a movement direction) associated with a leaf of a multi-leaf collimator, a position of a scanning table, an angle of the scanning table, a number (or count) of radiation fields (or sub-fields), a shape of a radiation field (or sub-field), an area of the radiation field (or sub-field), a perimeter of the radiation field (or sub-field), an angle of the radiation field, a deviation degree of the radiation field (or sub-field) from a beam center point, a ratio of an area to a perimeter of the radiation field (or sub-field), a number (or count) of unconnected areas in the radiation field, or the like, or any combination thereof, as described in connection with operation 520 in FIG. 5. In some embodiments, a sample parameter may include a plurality of sample sub-parameters. For example, the sample parameter may be a multi-leaf collimator parameter. The multi-leaf collimator parameter may include a plurality of sample sub-parameters including a position, a movement speed, a movement acceleration, and/or a movement direction associated with a leaf of the multi-leaf collimator.

In some embodiments, the plurality of sample plans may include a plurality of sample radiotherapy plans (e.g., a plurality of historical radiotherapy plans) of one or more candidate subjects (e.g., patients). The plurality of sample plans may correspond to different complexity levels within a preset complexity range. In some embodiments, the complexity level of the sample plan may be related to at least one sample parameter value in the sample plan. For example, the complexity level of the sample plan may be related to a size of the at least one sample parameter value in the sample plan, a change degree of the at least one sample parameter value in the sample plan, a change rate of the value of the at least one sample parameter value in the sample plan, or the like, or any combination thereof, as described in connection with operation 810 in FIG. 8.

In some embodiments, the data set may include at least one sample plan with a relatively high complexity level. In some embodiments, the data set may include at least one sample plan including at least one sample parameter with a relatively large sample parameter value. For example, the data set may include at least one sample plan including a number (or count) of sub-fields of which the value is relatively large. In some embodiments, the data set may include at least one sample plan including at least one sample parameter with a relatively small sample parameter value. For example, the data set may include at least one sample plan including an area of the sub-field of which the value is relatively small. In some embodiments, the data set may include at least one sample plan including at least one sample parameter with a relatively high change degree. For example, the data set may include at least one sample plan including a dose rate during the radiotherapy process of which the change degree is relatively high. In some embodiments, the data set may include at least one sample plan including at least one sample parameter with a relatively high change rate. For example, the data set may include at least one sample plan including a gantry angle during the radiotherapy process of which the change rate is relatively high.

In some embodiments, the data set may include at least one sample plan including at least one sample parameter, wherein the at least one sample plan is less possible to be involved. For example, the data set may include eccentric field plan and/or large field plan which is less possible to be applied.

In this way, a complexity level of the data set is greater than complexity levels of plans (e.g., radiotherapy plans) of almost all subjects (e.g., patients), that is, complexity levels of the plans (e.g., radiotherapy plans) of almost all subjects is within the complexity range of the data set. Accordingly, since a complexity level of a plan reflects the difficulty of a radiotherapy operation performed on a subject based on value (s) of radiotherapy parameter(s) in the plan, if a quality assurance test can be passed using a data set with a relatively high complexity level, it can be considered that the quality assurance test can also be passed using other plans (e.g., radiotherapy plans) with a relatively low complexity level. Therefore, for a period of time (e.g., a week, a month, a year, etc.), a quality assurance result can be obtained based on the data set. And then, before a target plan is performed, it can be determined whether to perform the target plan according to the quality assurance result, without performing another quality assurance test specifically for the target plan.

In some embodiments, the data set may be determined based on the parameter information (e.g., the limit performance information) of the at least one component of the medical device. The limit performance information of the at least one component may include the maximum movement speed of the at least one component, the maximum movement acceleration of the at least one component, a limit movement position of the at least one component, or the like, or any combination thereof, as described in connection with operation 610 in FIG. 6A. For example, the sample parameter of the data set may include at least one limit performance parameter of the at least one component of the medical device. With the parameter information (e.g., the limit performance information) of the at least one component of the medical device in the data set, a quality assurance test of the medical device may be performed based on the data set.

In some embodiments, the data set may be determined based on at least one sample plan with a relatively high complexity level and limit performance information of at least one component of the medical device. In this case, for a period of time (e.g., a week, a month, a year, etc.), both of a quality assurance result related to radiotherapy plans and a quality assurance result of the medical device can be obtained by performing a quality assurance test through directing the medical device to execute the data set (e.g., performing a radiotherapy operation based on value(s) of the sample parameter(s) in the data set). And then, before a target plan is performed, it can be determined whether to perform the target plan according to the quality assurance result related to the radiotherapy plans, without performing another quality assurance test specifically for the target plan.

In some embodiments, the data set may be determined based on the parameter information (e.g., the calculation limit of the test model) of the test model. For example, the sample parameter in the data set may include at least one calculation limit parameter of the test model. The test model may include an algorithm or a model that can determine a predicted test result based on sample parameter(s) in the data set. For example, the test model may include a dose model, a data simulation algorithm, or the like. More descriptions of the calculation limit of the test model (e.g., the dose model) may be found elsewhere in the present disclosure (e.g., FIG. 6A and descriptions thereof).

In some embodiments, the data set may be determined based on the plurality of sample plans, the limit performance information of the at least one component of the medical device, and the calculation limit of the test model. The sample parameter in the data set may include at least one radiotherapy parameter in the plurality of sample plans, at least one limit performance parameter of the at least one component of the medical device, and at least one calculation limit parameter of the test model.

In some embodiments, the data set may be a default setting when the medical device leaves a factory. In some embodiments, the data set may be manually determined by a user (e.g., a doctor) of the medical system 100, or one or more components (e.g., the processing device 120) of the medical system 100 according to an actual need.

In some embodiments, the data set may include a first data set, a second data set, a third data set, or the like, or any combination thereof, as described in connection with operation 610.

In 1420, the processing device 120 (e.g., the first determination module 1320) may determine an actual test result related to at least one of a target plan of a target subject or a medical device based on the data set.

The target plan may include at least one target parameter, each of which corresponds to at least one target parameter value. The target parameter may include a radiotherapy parameter associated with a radiotherapy process as described elsewhere in the present disclosure. In some embodiments, at least part of the at least one target parameter may have the corresponding sample parameter in the data set. In some embodiments, for the target parameter value of each of the at least one target parameter, the target parameter value may be within or outside the parameter range of the sample parameter corresponding to the target parameter.

In some embodiments, the target parameter value of each of the at least one target parameter may be within the parameter range of the sample parameter corresponding to the target parameter. For example, if a parameter range of a number (or count) of sub-fields in the data set is between 0 and 10, a number (or count) of sub-fields in the target plan needs to be within the parameter range of the number (or count) of sub-fields in the data set, to ensure that the complexity level of the data set is greater than the complexity level of the target plan.

In some embodiments, the processing device 120 may determine the actual test result by directing the medical device to execute the data set. The actual test result may include a test image (e.g., a radiation field image), a dose distribution (e.g., a measured dose distribution), or the like, or any combination thereof. More descriptions for determining the actual test result may be found elsewhere in the present disclosure (e.g., operation 620 in FIG. 6A and descriptions thereof).

In 1430, the processing device 120 (e.g., the second determination module 1330) may determine a quality assurance result related to the at least one of the target plan or the medical device based on the actual test result.

In some embodiments, the processing device 102 may determine a predicted test result based on the data set using the test model. The prediction test result may include an image (e.g., a simulated image), a dose distribution (e.g., a simulated dose distribution, a planned dose distribution), or the like, or any combination thereof. The processing device 120 may determine the quality assurance result based on the predicted test result and the actual test result. In the present disclosure, the quality assurance result may also be referred to as a data set execution result. For example, the actual test result may include a test image obtained by directing the medical device to execute the data set. The predicted test result may include a simulated image obtained based on the data set using the test model. The processing device 120 may determine the quality assurance result based on a difference between the test image and the simulated image. More descriptions for determining the quality assurance result may be found elsewhere in the present disclosure (e.g., operation 620 in FIG. 6A and descriptions thereof).

In some embodiments, the processing device 120 may determine whether a quality assurance test passes based on the quality assurance result. For example, the processing device 120 may determine whether the quality assurance test passes based on the difference between the test image and the simulated image. In response to determining that the difference between the test image and the simulated image is greater than a first difference threshold, the processing device 120 may determine that the quality assurance test does not pass. In response to determining that the difference between the test image and the simulated image is not greater than the first difference threshold, the processing device 120 may determine that the quality assurance test passes. As another example, the processing device 120 may determine whether the quality assurance test passes based on a difference between a measured dose distribution and a planned dose distribution.

In some embodiments, the processing device 120 may determine a plurality of actual test results (e.g., a plurality of test images) by directing the medical device to execute the same data set a plurality of times according to a preset test frequency. The preset test frequency may be set by a user (e.g., a doctor) of the medical system 100. For example, the preset test frequency may be once a day, once a week, once a month, once a year, or the like. Further, the processing device 120 may determine whether the quality assurance test passes based on the plurality of actual test results. For example, the processing device 120 may determine the quality assurance result based on a difference between a first test image obtained today and a second test image obtained yesterday. In response to determining that the difference between the first test image and the second test image is greater than a second difference threshold, the processing device 120 may determine that the quality assurance test does not pass. In response to determining that the difference between the first test image and the second test image is not greater than the second difference threshold, the processing device 120 may determine that the quality assurance test passes.

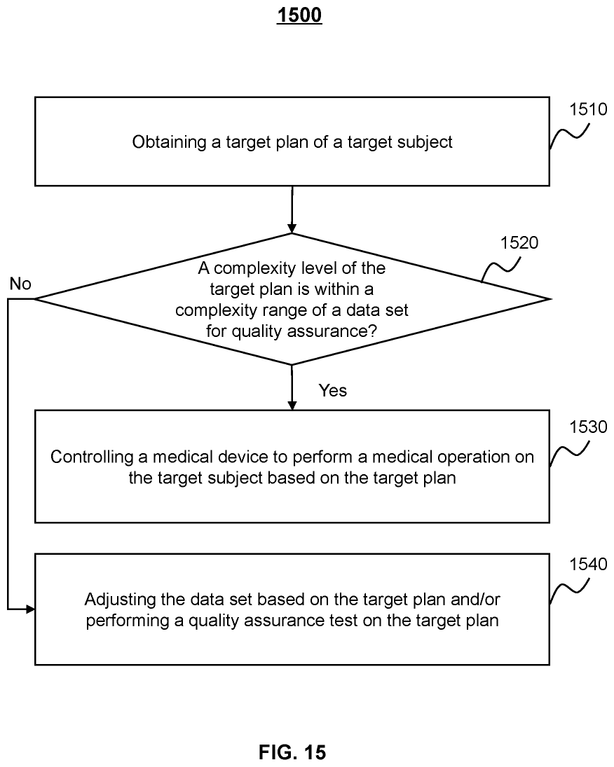

In response to determining that the quality assurance test passes, the processing device 120 may control the medical device to perform a medical operation on a target subject based on a target plan of the target subject. For example, the processing device 120 may control the at least one component of the medical device to perform a radiotherapy operation on the target subject based on value(s) of radiotherapy parameter(s) in the target plan of the target subject. In some embodiments, before the medical operation is performed on the target subject, the processing device 120 may determine whether a complexity level of the target plan is within the complexity range of the data set. More descriptions for determining whether the complexity level of the target plan is within the complexity range of the data set may be found elsewhere in the present disclosure (e.g., FIG. 15 and descriptions thereof).

In some embodiments, in response to determining that the quality assurance test passes, the processing device 120 may directly control the medical device to perform a medical operation on a target subject based on a target plan of the target subject, without determining whether a complexity level of the target plan is within the complexity range of the data set. In some embodiments, in the data set, if the number (or count) of the at least one sample parameter is greater than a type threshold, and/or the parameter range of each of the at least one sample parameter is greater than a range threshold, it may be indicated that the coverage of the data set is broad enough so that it is not necessary to determine whether a complexity level of the target plan is within the complexity range of the data set. In this case, in response to determining that the quality assurance test passes, the processing device 120 may omit the operation of determining whether a complexity level of the target plan is within the complexity range of the data set, and directly control the medical device to perform a medical operation on a target subject based on a target plan of the target subject. In some embodiments, the range threshold of each of the at least one sample parameter may be the same or different. In some embodiments, the type threshold and/or the range threshold may be a fixed value or adjustable based on the actual condition. For example, the type threshold and/or the range threshold may be set based on the frequency with which the medical device 110 performs radiotherapy plans. If the medical device 110 performs radiotherapy plans relatively frequently, the type threshold and/or the range threshold may be set as a relative large value, which indicates that a data set with a relatively high complexity level is needed to cover the complexity levels of plans performed by the medical device 110. If the medical device 110 performs radiotherapy plans occasionally, the type threshold and/or the range threshold may be set as a relative small value, which indicates that a data set with a relatively low complexity level is enough to cover the complexity levels of plans performed by the medical device 110.

In some embodiments, in response to determining that the quality assurance test does not pass, the processing device 120 may adjust value(s) of parameter(s) associated with the medical device and/or the sample parameter value(s) of sample parameter(s) in the data set. For example, at least one component of the medical device may be calibrated to improve a positioning accuracy of the at least one component and/or reduce a systematic error and/or a random error of the at least one component. As another example, the processing device 120 may adjust the sample parameter value(s) of the sample parameter(s) in the data set to adjust (e.g., decrease) the complexity level of the data set.

In some embodiments, in response to determining that the quality assurance test does not pass, the processing device 120 may determine a reason that the quality assurance test does not pass. For example, the processing device 120 may determine a reason that the quality assurance test does not pass based on a state of the medical device, the data set, and a prediction result using a second model as described elsewhere in the present disclosure (e.g., FIGS. 5-12 and descriptions thereof).

In some embodiments, the processing device 120 may generate a reminder. The reminder may be in the form of text, voice, a picture, a video, a haptic alert, or the like, or any combination thereof. In some embodiments, the reminder may include information regarding the quality assurance test result (whether the quality assurance test passes). In some embodiments, in response to determining that the quality assurance test does not pass, the reminder may include the reason that the quality assurance test does not pass and/or a recommended adjustment mode. For example, if the reason that the quality assurance test does not pass is that a rotation axis of a collimator is inconsistent with a beam axis, the reminder may include an offset between the rotation axis of the collimator and the beam axis. The recommended adjustment mode may be to adjust a position of the collimator and/or a position of a component of an accelerator to correct the rotation axis of the collimator and/or the beam axis. As another example, if the reason that the quality assurance test does not pass is that a measured dose distribution is inconsistent with a planned dose distribution, the reminder may include a difference between the measured dose distribution and the planned dose distribution. The recommended adjustment mode may be to adjust the beam energy and parameter(s) of component(s) (e.g., a collimator) of the medical device to ensure that the measured dose distribution is consistent with the planned dose distribution.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

In some embodiments, the data set may be updated with the development of radiotherapy technology and the change of application scenarios. In some embodiments, for different types of radiotherapy devices, the limit performance information of the radiotherapy devices may be different, and the types of parameters involved in the radiotherapy process using the radiotherapy devices may be different. Therefore, different data sets may be used in quality assurance tests for different types of radiotherapy devices. In some embodiments, the data set may include a plurality of data subsets. The plurality of data subsets may correspond to different quality assurance test frequencies and/or different types of plans. For example, a radiotherapy plan may be classified according to a type of a target area (e.g., a tumor) of the target subject, a type of a radiotherapy technology (e.g., a two-dimensional conformal radiotherapy, a three-dimensional conformal radiotherapy, an intensity-modulated radiotherapy, an image-guided radiotherapy, a bio-guided radiotherapy, a dose-guided radiotherapy, an online adaptive radiotherapy) used in the radiotherapy plan.

In some embodiments, since the change degrees of different types of parameters of the medical device over time are different, the checking frequency of different types of parameters of the medical device may be different. For example, for a parameter (e.g., an accelerator output, a positioning accuracy of a multi-leaf collimator) associated with the medical device that is easy to change, the checking frequency may be relatively high (e.g., once a day, once a week), to ensure the accuracy of the radiotherapy process. As another example, for a parameter (e.g., a positioning accuracy of a gantry rotation, a positioning accuracy of an isocenter of the medical device, a positioning accuracy of a scanning table) associated with the medical device that is not easy to change, the checking frequency may be relatively low (e.g., once a month, once a year), to reduce the time of the quality assurance test and improve the efficiency of the quality assurance test. In some embodiments, the data set may include a first data subset corresponding to an annual quality assurance test, a second data subset corresponding to a monthly quality assurance test, a third data subset corresponding to a weekly quality assurance test, and a fourth data subset corresponding to a daily quality assurance test. That is, in the annual quality assurance test, the quality assurance result may be determined by directing the medical device to execute the first data subset. In the monthly quality assurance test, the quality assurance result may be determined by directing the medical device to execute the second data subset. In the weekly quality assurance test, a quality assurance result may be determined by directing the medical device to execute the third data subset. In the daily quality assurance test, a quality assurance result may be determined by directing the medical device to execute the fourth data subset.

In some embodiments, types and/or numbers (or counts) of parameters of the medical device tested in the quality assurance tests based on the first data subset, the second data subset, the third data subset, and the fourth data subset may be different. For example, the parameter associated with the medical device that is not easy to change may be tested in the quality assurance tests using the first data subset and the second data subset. As another example, the parameter associated with the medical device that is easy to change may be tested in the quality assurance tests using the third data subset and the fourth data subset. As still another example, a relatively large number of parameters associated with the medical device may be tested in the quality assurance tests using the first data subset and the second data subset. As still another example, a relatively small number of parameters associated with the medical device may be tested in the quality assurance tests using the third data subset and the fourth data subset.

FIG. 15 is a flowchart illustrating an exemplary process for performing a quality assurance test according to some embodiments of the present disclosure. In some embodiments, process 1500 may be executed by the medical system 100. For example, the process 1500 may be implemented as a set of instructions (e.g., an application) stored in a storage device (e.g., the storage device 130, the storage device 220, and/or the storage 390). In some embodiments, the processing device 120 (e.g., the processor 210 of the computing device 200, the CPU 340 of the mobile device 300, and/or one or more modules illustrated in FIG. 13) may execute the set of instructions and may accordingly be directed to perform the process 1500. The operations of the illustrated process presented below are intended to be illustrative. In some embodiments, the process 1500 may be accomplished with one or more additional operations not described and/or without one or more of the operations discussed. Additionally, the order of the operations of process 1500 illustrated in FIG. 15 and described below is not intended to be limiting.

In 1510, the processing device 120 (e.g., the obtaining module 1310) may obtain a target plan of a target subject.

Operation 1510 may be performed in a similar manner as operation 520 as described in connection with FIG. 5, the descriptions of which are not repeated here.

In 1520, the processing device 120 (e.g., the second determination module 1330) may determine whether a complexity level of the target plan is within a complexity range of a data set for quality assurance.

In some embodiments, the processing device 120 may determine whether a sample parameter value of a sample parameter (e.g., a number (or count) of sub-fields) in the data set is greater than or equal to a target parameter value of a corresponding target parameter (e.g., a number (or count) of sub-fields) in the target plan. In response to determining that the sample parameter value of the sample parameter (e.g., a number (or count) of sub-fields) in the data set is greater than or equal to the target parameter value of the corresponding target parameter (e.g., a number (or count) of sub-fields) in the target plan, the processing device 120 may determine that the complexity level of the target plan is within the complexity range of the data set.

In some embodiments, the processing device 120 may determine whether a sample parameter value of a sample parameter (e.g., an area of a sub-field) in the data set is less than or equal to a target parameter value of a corresponding target parameter (e.g., an area of a sub-field) in the target plan. In response to determining that the sample parameter value of the sample parameter (e.g., an area of a sub-field) in the data set is less than or equal to the target parameter value of the corresponding target parameter (e.g., an area of a sub-field) in the target plan, the processing device 120 may determine that the complexity level of the target plan is within the complexity range of the data set.

In some embodiments, the processing device 120 may determine whether a change degree of a sample parameter value of a sample parameter (e.g., a dose rate) in the data set is higher than or equal to a change degree of a target parameter value of a corresponding target parameter (e.g., a dose rate) in the target plan. In response to determining that the change degree of the sample parameter value of the sample parameter (e.g., a dose rate) in the data set is higher than or equal to the change degree of the target parameter value of the corresponding target parameter (e.g., a dose rate) in the target plan, the processing device 120 may determine that the complexity level of the target plan is within the complexity range of the data set.

In some embodiments, the processing device 120 may determine whether a change rate of a sample parameter value of a sample parameter (e.g., a gantry angle) in the data set is higher than or equal to a change rate of a target parameter value of a corresponding target parameter (e.g., a gantry angle) in the target plan. In response to determining that the change rate of the sample parameter value of the sample parameter (e.g., a gantry angle) in the data set is higher than or equal to the change rate of the target parameter value of the corresponding target parameter (e.g., a gantry angle) in the target plan, the processing device 120 may determine that the complexity level of the target plan is within the complexity range of the data set.

In some embodiments, in response to determining that sample parameter values of all sample parameters, change degrees of sample parameter values of all sample parameters, and change rates of sample parameter values of all sample parameters in the data set can cover (e.g., greater than, equal to, or less than according to a type of the sample parameter) target parameter values of corresponding target parameters respectively, change degrees of target parameter values of corresponding target parameters respectively, and change rates of target parameter values of corresponding target parameters respectively in the target plan, the processing device 120 may determine that the complexity level of the target plan is within the complexity range of the data set.

In some embodiments, a user (e.g., a doctor) of the medical system 100 or the processing device 120 may select one or more sample parameters according to an actual need and/or an importance of the sample parameter. In response to determining that sample parameter value(s) of selected sample parameter(s), change degree(s) of sample parameter value(s) of selected sample parameter(s), and/or change rate(s) of sample parameter value(s) of selected sample parameter(s) in the data set can cover (e.g., greater than, equal to, or less than according to a type of the sample parameter) target parameter value(s) of corresponding target parameter(s) respectively, change degree(s) of target parameter value(s) of corresponding target parameter(s) respectively, and change rate(s) of target parameter value(s) of corresponding target parameter(s) respectively in the target plan, the processing device 120 may determine that the complexity level of the target plan is within the complexity range of the data set.

In some embodiments, in response to determining that a ratio of sample parameter value(s) of sample parameter(s) in the data set that can cover (e.g., greater than, equal to, or less than according to a type of the sample parameter) target parameter value(s) of corresponding target parameter(s) in the target plan respectively, to a total number (or count) of target parameter(s) in the target plan is greater than a ratio threshold (e.g., 90%, 95%, 99%), the processing device 120 may determine that the complexity level of the target plan is within the complexity range of the data set.

In some embodiments, to determine whether the complexity level of the target plan is within the complexity range of the data set, the processor device 120 may determine whether the type of the at least one target parameter is covered by the data set, and/or determine, for each of the at least one target parameter, whether the at least one target value of the target parameter is within the parameter range of the sample parameter corresponding to the target parameter.

In 1530, in response to determining that the complexity level of the target plan is within the complexity range of the data set, the processing device 120 (e.g., the second determination module 1330) may control the medical device to perform a medical operation on the target subject based on the target plan.

In some embodiments, in response to determining that the complexity level of the target plan is within the complexity range of the data set, that is, the complexity level of the target plan is lower than the complexity level of the data set, it may indicate that if the quality assurance test based on the data set with a relatively high complexity level can pass, the quality assurance test based on the target plan with a relatively low complexity level can also pass. For example, in response to determining that the complexity level of the target plan is within the complexity range of the data set, the processing device 120 may control the at least one component of the medical device to perform a radiotherapy operation on the target subject based on value(s) of radiotherapy parameter(s) in the target plan of the target subject.

In 1540, in response to determining that the complexity level of the target plan is not within the complexity range of the data set, the processing device 120 (e.g., the second determination module 1330) may adjust the data set based on the target plan, and/or perform a quality assurance test on the target plan.

In some embodiments, in response to determining that the complexity level of the target plan is not within the complexity range of the data set, that is, the complexity level of the target plan is higher than the complexity level of the data set, it may indicate that even though the quality assurance test based on the data set with a relatively low complexity level can pass, the quality assurance test based on the target plan with a relatively high complexity level may not pass.

In some embodiments, in response to determining that the complexity level of the target plan is not within the complexity range of the data set, the processing device 120 may adjust the sample parameter value(s) of the sample parameter (s) to adjust (e.g., decrease) the complexity range of the data set. For example, the processing device 120 may determine the target parameter value(s) of the target parameter(s) in the target plan as the sample parameter value(s) of the corresponding sample parameter (s) in the data set.

In some embodiments, in response to determining that the complexity level of the target plan is not within the complexity range of the data set, the processing device 120 may adjust the target parameter value(s) of the target parameter(s) in the target plan. For example, the processing device 120 may adjust the target parameter value(s) of the target parameter(s) in the target plan to adjust (e.g., decrease) the complexity level of the target plan.

In some embodiments, in response to determining that the complexity level of the target plan is not within the complexity range of the data set, a quality assurance test may be performed on the target plan by directing the medical device to execute the target parameter value(s) of the target parameter(s) in the target plan. For example, the processing device 120 may determine an actual test result by directing the medical device to execute the target plan. The processing device 120 may determine a predicted test result based on the target plan using the test model. The processing device 120 may determine a quality assurance result based on the predicted test result and the actual test result. The processing device 120 may determine whether the quality assurance test passes based on the quality assurance result. In response to determining that the quality assurance test does not pass, the processing device 120 may adjust the target parameter value(s) of the target parameter(s) in the target plan and/or value(s) of parameter (s) associated with the medical device until the quality assurance test passes. In response to determining that the quality assurance test passes, the processing device 120 may control the medical device to perform the medical operation on the target subject based on the target plan.

In some embodiments, before the medical operation is performed on the target subject, a verification device may be used to verify identity information of the target subject, positioning information of the target subject, a state of at least one component of the medical device, or the like, or any combination thereof. In some embodiments, the verification device may include a face recognition device, a fingerprint recognition device, a voice recognition device, a barcode scanning device, a sensing chip, a camera, a sensor (e.g., a speed sensor, an acceleration sensor), an augmented reality (AR) device (AR), a virtual reality (VR) device, a hybrid reality (MR) device, or the like, or any combination thereof.

The identity information of the target subject may include an ID number, a name, the sex, the age, a date of birth, or the like, or any combination thereof, of the target subject. In some embodiments, feature information (e.g., face feature information, fingerprint feature information, voice feature information) of the target subject may be obtained using the verification device (e.g., the face recognition device, the fingerprint recognition device, the voice recognition device, the camera), and the identity information of the target subject may be verified based on the feature information of the target subject. In some embodiments, the identity information of the target subject may be verified based on a unique identifier associated with the target subject. The unique identification may include a barcode, a quick response (QR) code, a serial number including letters and/or numbers, or the like, or any combination thereof. For example, the identity information of the target subject may be verified by scanning a barcode on a hand band of the target subject using the verification device (e.g., the camera, the barcode scanning device).

The positioning information of the target subject may include position information and/or posture information of the target subject. In some embodiments, image data of the target subject may be obtained by the verification device (e.g., a camera). The processing device 120 may identify the positioning information of the target subject based on the image data of the target subject. In some embodiments, the processing device 120 may determine whether the positioning information of the target subject needs to be adjusted. For example, the processing device 120 may determine whether the positioning information of the target subject needs to be adjusted by comparing the positioning information of the target subject with standard positioning information. The standard positioning information may include a standard position that the target subject needs to be located in, and a standard posture that the target subject needs to hold during a scan to be performed on the target subject. In some embodiments, the processing device 120 may determine the standard positioning information based on the target plan of the target subject. For example, a user (e.g., a doctor, a technician) of the medical system 100 may determine the standard positioning information based on the target plan of the target subject, and store the standard positioning information in a storage device (e.g., storage device 130) of the medical system 100. The processing device 120 may obtain the standard positioning information from the storage device (e.g., storage device 130). In response to determining that the positioning information of the target subject is inconsistent with the standard positioning information, the processing device 120 may generate a reminder to the user and/or the target subject to instruct the target subject to adjust his/her position and/or posture.

The state of the at least one component of the medical device may include position information of the at least one component, movement information (e.g., a movement speed, a movement acceleration) of the at least one component, or the like, or any combination thereof. In some embodiments, the state of the medical device may also include beam and dose information, positioning accuracy information of at least one component (e.g., a gantry, a radiation source, a collimator) of the medical device, operation error information of the at least one component of the medical device, a data set execution result, or the like, or any combination thereof, as described elsewhere in the present disclosure (e.g., operation 510 in FIG. 5). The state of the at least one component of the medical device may indicate whether the at least one component of the medical device is in a normal working state.

In some embodiments, the verification device (e.g., a position encoder, the speed sensor, the acceleration sensor) may be installed on the at least one component (e.g., a scanning table, a gantry) of the medical device, and actual state information (e.g., an actual position, an actual movement speed, an actual movement acceleration) of the at least one component may be detected by the verification device. The processing device 120 may obtain expected state information of the at least one component. The expected state information (e.g., an expected position, an expected movement speed, an expected movement acceleration) of the at least one component may be set by a user (e.g., a doctor, a technician) of the medical system 100 via a control device (e.g., the terminal device 140) of the medical device. The processing device 120 may determine whether the state of the at least one component of the medical device satisfies a preset condition by comparing the actual state information of the at least one component and the expected state information of the at least one component. For example, the processing device 120 may determine whether a speed accuracy of the at least one component satisfies a preset condition by determining whether a difference between the actual movement speed of the at least one component and the expected movement speed of the at least one component is less than a speed threshold. In response to determining that the difference between the actual movement speed of the at least one component and the expected movement speed of the at least one component is less than the speed threshold, the processing device 120 may determine that the speed accuracy of the at least one component satisfies the preset condition. As another example, the processing device 120 may determine whether a position accuracy of the at least one component satisfies a preset condition by determining whether a difference between the actual position of the at least one component and the expected position of the at least one component is less than a position threshold. In response to determining that the difference between the actual position of the at least one component and the expected position of the at least one component is less than the position threshold, the processing device 120 may determine that the position accuracy of the at least one component satisfies the preset condition. More descriptions for determining whether the state of the medical device satisfies a preset condition may be found elsewhere in the present disclosure (e.g., FIG. 5 and descriptions thereof). In response to determining that the state of the at least one component of the medical device satisfies the preset condition, it may indicate that the at least one component of the medical device is in the normal working state, and the medical device may be used to perform the medical operation on the target subject. In response to determining that the state of the at least one component of the medical device does not satisfy the preset condition, it may indicate that at least one component of the medical device is in a unnormal working state, and the medical device may need to be corrected.

According to some embodiments of the present disclosure, by using a data set that is not specific to a specific patient and the complexity level of the data set is greater than plans of almost all patients, the quality assurance test disclosed in the present disclosure can replace traditional quality assurance tests for the medical device and/or the plan of each patient. For example, in response to determining that the quality assurance test passes according to the quality assurance result determined based on the data set, the medical operation (e.g., the radiotherapy operation) may be performed on the target subject directly based on the target plan of the target subject using the medical device. That is, it is not necessary to perform a quality assurance test for the target plan of the target patient before the medical operation of the target subject, which can save the time of the radiotherapy process, improve the efficiency of the radiotherapy process, and simplify the radiotherapy process. In some embodiments, before the medical operation is performed on the target object, a determination may be made as whether the complexity level of the target plan is within the complexity range of the data set, which can further guarantee the feasibility of the target plan and the quality of the radiotherapy process. In addition, the data set may be updated according to different test frequencies and/or different types of radiotherapy plans. The data set may also be updated with the development of radiotherapy technology and the change of application scenarios, so as to ensure the rationality and accuracy of the quality assurance test based on the data set. Furthermore, before the medical operation is performed on the target subject, the identity information of the target subject, the positioning information of the target subject, and/or the state of at least one component of the medical device may be verified using the verification device, which can ensure that the medical device is in a normal working state, and the quality of the radiotherapy process may further be improved.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the system and method for quality assurance disclosed in the present disclosure may be applied to a medical device or a non-medical device. Accordingly, the target plan may be a medical plan or a non-medical plan. The medical plan may include an imaging plan or a treatment plan (e.g., a radiotherapy plan).Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python or the like, conventional procedural programming languages, such as the “C” programming language, Visual Basic, Fortran 2003, Peri, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

What is claimed is:

1. A method for quality assurance, which is implemented on a computing device including at least one processor and at least one storage device, the method comprising:
obtaining a state of a medical device;
obtaining a target plan of a target subject;
determining a prediction result based on the state of the medical device and the target plan of the target subject; and
determining whether a quality assurance test passes based on the prediction result.

2. The method of claim 1, wherein the state of the medical device includes at least one of beam information corresponding to a plurality of dose rates, positioning accuracy information of at least one component of the medical device, or operation error information of the at least one component of the medical device.

3. The method of claim 1, wherein the obtaining a state of a medical device comprises:
obtaining a data set, wherein the data set is determined based on at least one of at least one candidate plan, limit performance information of at least one component of the medical device, or a calculation limit of a dose model; and
obtaining the state of the medical device by directing the medical device to execute the data set.

4. The method of claim 3, wherein
the data set includes at least one of a first data set, a second data set, or a third data set; and
the obtaining a data set comprises at least one of:
determining the first data set based on at least one first candidate plan, wherein a value of a first candidate parameter in the at least one first candidate plan is within a first range;
determining the second data set based on at least one second candidate plan, wherein a value of a second candidate parameter in the at least one second candidate plan is outside a second range; or
determining the third data set based on at least one of the limit performance information of the at least one component of the medical device or the calculation limit of the dose model.

5. The method of claim 3, wherein
the data set includes at least one sample parameter, each of which corresponds to at least one sample parameter value; and
the obtaining the state of the medical device by directing the medical device to execute the data set comprises:
determining an actual test result related to at least one of the target plan of the target subject or the medical device by directing the medical device to execute the data set, the target plan including at least one target parameter, each of which corresponds to at least one target parameter value;
determining a data set execution result based on the actual test result; and
obtaining the state of the medical device based on the data set execution result.

6. The method of claim 1, wherein the determining a prediction result based on the state of the medical device and the target plan of the target subject comprises:
determining at least one of feature information related to a complexity level of the target plan or a target fluence map based on the target plan of the target subject; and
determining the prediction result based on the state of the medical device and the at least one of the feature information related to the complexity level of the target plan or the target fluence map.

7. The method of claim 1, wherein
the target subject includes a plurality of regions of interest (ROIs);
the prediction result includes dose distributions corresponding to the plurality of ROIs respectively; and
the determining whether a quality assurance test passes based on the prediction result comprises:
determining a weight corresponding to each ROI of the plurality of ROIs; and
determining whether the quality assurance test passes based on the weights and the dose distributions corresponding to the plurality of ROIs respectively.

8. The method of claim 1, wherein the determining a prediction result based on the state of the medical device and the target plan of the target subject comprises:
determining the prediction result based on the state of the medical device and the target plan of the target subject using a first model, wherein
the first model is a machine learning model; and
the prediction result includes at least one of a predicted image of the target subject, a gamma passing rate, or a dose distribution.

9. The method of claim 8, wherein the method further comprises:
in response to determining that the quality assurance test does not pass based on the prediction result, determining a reason that the quality assurance test does not pass based on the state of the medical device, the target plan of the target subject, and the prediction result using a second model.

10. The method of claim 9, wherein the method further comprises:
adjusting, based on the reason that the quality assurance test does not pass, a value of a parameter associated with at least one of the medical device, the target plan, or a dose model.

11. The method of claim 10, wherein the method further comprises:
determining an updated prediction result based on an adjusted value of the parameter using the first model;
determining whether the quality assurance test passes based on the updated prediction result; and
in response to determining that the quality assurance test passes, controlling the medical device to treat or scan the target subject according to an updated target plan, wherein the updated target plan is determined based on the adjusted value of the parameter.

12. A method for quality assurance, which is implemented on a computing device including at least one processor and at least one storage device, the method comprising:
obtaining a state of a medical device;
obtaining a target plan of a target subject;
determining a prediction result based on the state of the medical device and the target plan of the target subject using a quality assurance model, wherein the quality assurance model is a machine learning model.

13. A method for quality assurance, which is implemented on a computing device including at least one processor and at least one storage device, the method comprising:
obtaining a data set for quality assurance, the data set including at least one sample parameter, each of which corresponds to at least one sample parameter value;
determining an actual test result related to at least one of a target plan of a target subject or a medical device based on the data set, the target plan including at least one target parameter related to the at least one sample parameter, each of which corresponds to at least one target parameter value; and
determining a quality assurance result related to the at least one of the target plan or the medical device based on the actual test result.

14. The method of claim 13, wherein the determining a quality assurance result related to the at least one of the target plan or the medical device based on the actual test result comprises:
determining a predicted test result based on the data set using a test model; and
determining the quality assurance result based on the predicted test result and the actual test result.

15. The method of claim 14, wherein
the actual test result includes a test image obtained by the medical device based on the data set;
the predicted test result includes a simulated image obtained based on the test model; and
the determining the quality assurance result based on the predicted test result and the actual test result comprises:
determining the quality assurance result based on a difference between the test image and the simulated image.

16. The method of claim 13, wherein the data set is determined based on at least one of a plurality of sample plans or limit performance information of at least one component of the medical device.

17. The method of claim 16, wherein the plurality of sample plans comprise plans corresponding to different complexity levels.

18. The method of claim 17, further comprising:
determining whether a complexity level of the target plan is within a complexity range of the data set, the complexity range of the data set being determined based on the complexity level of the at least one of the plurality of sample plans corresponding to the data set; and
in response to determining that the complexity level of the target plan is within the complexity range, controlling the medical device to perform a medical operation on the target subject based on the target plan; or
in response to determining that the complexity level of the target plan is not within the complexity range, proceeding with at least one of:
adjusting the data set based on the target plan, or
performing a quality assurance test on the target plan.

19. The method of claim 13, wherein
the data set include a plurality of data subsets, and
the plurality of data subsets is configured to correspond to at least one of: different quality assurance test frequencies or different types of plans.

20. The method of claim 13, further comprising:
determining whether a quality assurance test passes based on the quality assurance result; and
in response to determining that the quality assurance test does not pass, adjusting at least one of a parameter of the medical device or the sample parameter value of the data set.
